(19) United States
(12) Patent Application Publication
(10) Pub. No.: US 2010/0048482 A1
(43) Pub. Date: Feb. 25, 2010

(54) COMPOSITIONS AND METHODS FOR MODULATING EPSILON PROTEIN KINASE C-MEDIATED CYTOPROTECTION

(75) Inventors: Daria Mochly-Rosen, Menlo Park, CA (US); Grant R. Budas, Menlo Park, CA (US)

Correspondence Address: King & Spalding LLP, P.O. Box 889, Belmont, CA, 94002-0889, US

(73) Assignee: Board of Trustees of the Leland Stanford Junior University, Stanford, CA (US)

(21) Appl. No.: 12/542,567

(22) Filed: Aug. 17, 2009

Related U.S. Application Data
(60) Provisional application No. 61/089,236, filed on Aug. 15, 2008.

(51) Int. Cl. A61K 38/16; C07K 7/06; A61K 38/10; A61K 38/08; A61P 3/00

(52) U.S. Cl. 514/12; 530/329; 514/16; 514/17; 514/15; 514/14; 514/13

(57) ABSTRACT

Compositions and methods for reducing ischemic cell damage and treating mitochondrial disorders using therapeutic agents derived from the V2 domain of epsilon protein kinase C (PKC) are described.

### CROSS-REFERENCE TO RELATED APPLICATIONS

This application claims priority under 35 U.S.C. 119(e) to U.S. Provisional Application No. 61/089,236, filed on Aug. 15, 2008, which is hereby incorporated by reference.

### STATEMENT REGARDING GOVERNMENT INTEREST

This was made with Government support under contract AA11147 awarded by the National Institutes of Health. The Government has certain rights in this invention.

### REFERENCE TO SEQUENCE LISTING, TABLE OR COMPUTER PROGRAM

A Sequence Listing is being submitted electronically via EFS in the form of a text file, created Aug. 17, 2009, and named “586008265US00seq.txt” (41701 bytes), the contents of which are incorporated herein by reference in their entirety.

### TECHNICAL FIELD

The subject matter described herein relates to compositions and methods for modulating ischemic cell damage and treating mitochondrial disorders using therapeutic agents derived from the epsilon protein kinase C (εPKC) isozyme.

### BACKGROUND

Mitochondria are organelles present in eukaryotic cells that provide energy for cellular activities through oxidative phosphorylation. Mitochondria are also involved in intracellular signaling and regulate both necrotic and apoptotic cell death, (Newmeyer, D. D. and S. Ferguson-Miller, Cell. 2003. 112:481-90; Rasola, A. and P. Bernardi, Apoptosis, 2007. 12:815-33, and Halestrap, A. P., Biochem Soc Trans, 2006. 34:232-7) suggesting a role for mitochondria in the pathophysiology of human diseases such as Parkinson's, Alzheimers, diabetes, and ischemic heart disease (DiMauro, S. and E. A. Schon, N Engl J Med, 2003. 348:2656-68). An increasing number of mitochondrial kinases, phosphatses, and phosphoproteins have been described, suggesting that reversible phosphorylation is important in mitochondrial function (Pagliarini, D. J. and J. E. Dixon, Trends Biochem Sci, 2006. 31:26-34; Horbinski, C. and C. T. Chu, Free Radic Biol Med, 2005, 38:2-11).

The epsilon isozyme of protein kinase C (εPKC) is known to play a role in cell survival, particularly in endogenous cytoprotection. εPKC is central to the phenomenon of ischemic preconditioning, which reduces cellular damage during reperfusion (Chen, C. H. et al., PNAS, 1999. 96:12784-12789, Liu, G. S. et al., JMCC, 1999. 31: 1937-1948). While εPKC is a cytosolic, rather than a mitochondrial protein, some εPKC substrates, particularly aldehyde dehydrohenase 2 (ALDH2) (Chen C. H. et al., Science 2008. 321: 1493-5), cytochrome c oxidase (COIV) (Ogbi, M., et al., Biochem J, 2004. 382:923-32), and components of the mitochondrial permeability transition pore (MPTP) (Baines, C. P., et al, Circ Res, 2002. 90:390-7; Ping, P., et al. Circ Res, 2001. 88:59-62) have been localized to the mitochondria and mitochondrial targets of εPKC may have cardioprotective properties(Baines, 2002: Baines, 2003; Jaburek, M., et al., Circ Res, 2006. 99:878-83; Agnetti, G., et al., Pharmacol Res, 2007. 55:511-22; and Lawrence, K. M., et al., Biochem Biophys Res Commun, 2004. 321:479-86). Studies have demonstrated that εPKC can translocate to cardiac mitochondria (Budas, G. R. and D. Mochly-Rosen, Biochem Soc Trans, 2007. 35:1052-4; Ohnuma, Y., et al., Am J Physiol Heart Circ Physiol, 2002. 283:H440-7; Ogbi, M., et al., Biochem J, 2004. 382:923-32; and Lawrence et al., Biochem Biophys Res Commun, 2004. 321:479-86) and εPKC has been associated with mitochondrial K_{ATP} channel activity. εPKC has also been shown to mediate the protective response of the myocardium to thermal preconditioning (Joyeux, M., et al., J Mol Cell Cardiol, 1997. 29:3311-9).

HSP90 is a ubiquitously expressed protein chaperone involved in protein folding (Pearl, L. H. and C. Prodromou, Annu Rev Biochem, 2006. 75:271-94). HSP90 has been reported to have cardioprotective effects that confer increased resistance to ischemia-reperfusion injury(Kupatt, C., et al., Arterioscler Thromb Vasc Biol, 2004. 24:1435-41; Marber, M. S., et al., J Clin Invest, 1995. 95:1446-56; Morris, S. D., et al., J Clin Invest, 1996. 97:706-12; Griffin, T. M., T. V. Valdez, and R. Mestril, Am J Physiol Heart Circ Physiol, 2004. 287:H1081-8; Brar, B. K., et al., J Endocrinol, 2002. 172:283-93; and Shi, Y., et a., Circ Res, 2002. 91:300-6. Conversely, the inhibition of HSP90 has been shown to exacerbate ischemia/reperfusion (IR) injury (Boengler, K., et al. Cardiovasc Res, 2005. 67:234-44) and abolish cardioprotection induced by ischemic preconditioning (Piper, H. M., Y. Abdallah, and C. Schafer, Cardiovasc Res, 2004. 61:365-71). While the primary cytoprotective function of HSP90 in thought to be the removal of misfolded proteins (Latchman, D. S., Cardiovasc Res, 2001. 51:637-46), recent studies have suggested that HSP90 plays a role in the mitochondrial import of proteins (Young, J. C., N. J. Hoogenraad, and F. U. Hartl, Cell, 2003. 112:41-50), including proteins involved in cardioprotective signaling (Rodriguez-Sinovas, A., et al., Circ Res, 2006. 99:93-101; Jiao, J. D., et al., Cardiovasc Res, 2008. 77:126-33).

### REFERENCES

The references cited herein are hereby incorporated by reference in their entirety.

### BRIEF SUMMARY

The following aspects and embodiments thereof described and illustrated below are meant to be exemplary and illustrative, not limiting in scope.

In one aspect, a peptide consisting of an amino acid sequence that is at least 80% identical to the amino acid sequence of P-K-D-N-E-E-R (SEQ ID NO: 1) is provided. In some embodiments, the peptide is at least 90% identical to the amino acid sequence of P-K-D-N-E-E-R (SEQ ID NO: 1). In particular embodiments, the peptide is at least 95% identical to the amino acid sequence of P-K-D-N-E-E-R (SEQ ID NO: 1). In some embodiments, the peptide is attached to a carrier to facilitate transport through a cell membrane or into a mitochondria.

In some embodiments, the peptide modulates mitochondrial import.

In a related aspect, a pharmaceutical composition comprising the peptide and a suitable pharmaceutical excipient is provided.

In another related aspect, a peptide consisting of a sequence of amino acids having at least 80% sequence identity to a contiguous sequence of between 5-15 amino acids residues of the V2 region of epsilon-PKC is provided.

In some embodiments, the peptide modulates mitochondrial translocation of εPKC. In some embodiments, the peptide activates mitochondrial translocation of εPKC. In some embodiments, the peptide inhibits mitochondrial translocation of εPKC.

In some embodiments, the peptide is attached to a carrier to facilitate transport through a cell membrane or into a mitochondria.

In a further aspect, a method for treating a mitochondria-related disorder in a subject is provided. The method comprises administering to the subject an isolated peptide having a sequence of amino acid residues corresponding to a contiguous sequence of amino acid residues from the V2 region of epsilon PKC. Administration of the peptide modulates translocation of epsilon-PKC to the mitochondria, thereby reducing symptoms of the mitochondria-related disorder.

In a further aspect, a method for reducing cell damage following ischemic reperfusion is provided. The method comprises administering to the subject an isolated peptide having a sequence of amino acid residues corresponding to a contiguous sequence of amino acid residues from the V2 region of epsilon PKC. Administration of the peptide modulates translocation of epsilon-PKC to the mitochondria, thereby reducing cell damage.

In a further aspect, a method for reducing cell damage mediated by HSP90 is provided. The method comprises administering to the subject an isolated peptide having a sequence of amino acid residues corresponding to a contiguous sequence of amino acid residues from the V2 region of epsilon PKC. Administration of the peptide modulates translocation of epsilon-PKC to the mitochondria, thereby reducing cell damage.

In a further aspect, a method for treating a mitochondria-related disorder in a subject is provided. The method comprises administering to the subject an isolated peptide having a sequence of amino acid residues corresponding to a contiguous sequence of amino acid residues from the V2 region of epsilon PKC. Administration of the peptide modulates the HSP90-dependent translocation of epsilon-PKC to the mitochondria, thereby reducing symptoms of the mitochondria-related disorder.

In a further aspect, a method for modulating interactions between epsilon-PKC and HSP90 in mitochondria is provided. The method comprises incubating the mitochondria in the presence of a peptide with a sequence of amino acid residues corresponding to a contiguous sequence of amino acid residues from the V2 region of epsilon PKC. Incubating modulates intermolecular interactions between epsilon-PKC and HSP90.

In yet a further aspect, a method for modulating mitochondrial import is provided, comprising, incubating the mitochondria in the presence of a peptide comprising a contiguous portion of the V2 region of epsilon PKC, wherein the incubating modulates mitochondrial import of a cytosolic polypeptide. In particular embodiments, the cytosolic polypeptide is epsilon-PKC.

In some embodiments, the peptide used in the methods is a sequence of amino acids having at least 80% sequence identity to a contiguous sequence of between 5-15 amino acids residues of the V2 region of epsilon-PKC. In a particular embodiment, the peptide has the amino acid sequence of SEQ ID NO: 1. The peptide may be attached to a carrier to facilitate transport through a cell membrane or into a mitochondria.

In addition to the exemplary aspects and embodiments described above, further aspects and embodiments will become apparent by reference to the drawings and by study of the following descriptions.

### BRIEF DESCRIPTION OF THE DRAWINGS

FIG. 1A is an illustration representing normoxia and ischemia-reperfusion (IR) treatment regimens used herein to determine whether HSP90 contributes to the endogenous protective response of the myocardium during reperfusion following ischemic event.

FIG. 1B is a graph of creatine phosphokinase (CPK) levels in units/l in the buffer perfusate for IR in the presence and absence of geldanamycin (GA), and in the absence and presence of GA after normoxia.

FIG. 1C shows immunoblots of εPKC, δPKC, ANT (a marker of mitochondrial fraction) and GAPDH, (a marker of cytosolic fraction) levels, after normoxia (Norm) (left lanes), after ischemia/reperfusion (IR) in the absence of GA (middle lane), and after IR in the presence of GA (right lane) in the mitochondria (upper three panels) and in the cytosol (lower two panels).

FIGS. 1D-1E show the translocation of εPKC or δPKC, respectively, expressed as the percentage of isozyme in the mitochondrial fraction over the amount of isozyme in non-treated cells (Norm), for cells treated as indicated in FIG. 1C.

FIGS. 2A-2D are immunoblots from SDS-PAGE gels showing the results of co-immunoprecipitation experiments performed using the indicated antibodies for immunoprecipitation (IP) followed by the indicated antibodies for immunoblot (western blot (WB)) analysis. The gels show the results for beads alone, during normoxia, after IR, and after IR in the presence of GA, FIG. 2A shows the results for the cytosolic fraction while FIGS. 2B-2D show the results for mitochondrial fractions.

FIG. 3A shows computer-generated electron micrographs showing immunostaining of mitochondrial sections with εPKC-specific antibodies followed by gold-conjugated secondary antibody during normoxia (upper left), after ischemia and reperfusion (IR, upper right), after IR in the presence of GA (lower left), and of a mitochondrial section treated with the gold-conjugated, secondary antibody alone control (lower right),

FIG. 3B is a graph quantifying the mitochondrial δPKC (gold particles/mitochondria) as observed in the electron micrographs of FIG. 3A, showing the mitochondrial εPKC during normoxia (Norm), and after IR in the presence (+) and absence (−) of GA.

FIG. 3C is an illustration showing a method for preparing mitochondrial subfractions.

FIG. 3D are SDS-PAGE gel immunoblots of the mitochondrial sub-fractions, inner mitochondrial membrane (IMM) and matrix, probed with εPKC-specific antibodies, ANT (a marker of IMM), Grp75 (a marker of matrix fraction), and enolase (a cytosolic marker) under normoxia, IR, and IR in the presence of GA.

FIG. 3E is a graph of the percentage of PKCε in IMM under normoxia and IR in the presence (+) and absence (−) of GA.

FIG. 3F shows SDS-PAGE gel immunoblots of sub-mitochondrial particles treated with high pH, high salt, or trypsin (at 0, 5, 10, and 20 minutes) and labeled with antibodies specific for εPKC, adenine nucleotide translocase (ANT), or cytochrome c (Cyt c).

FIG. 4A shows a homologous sequence between residues 139-145 of εPKC and residues 552-558 of HSP90, with the location of the homologous sequences indicated on protein schemes and shown by cross-hatching.

FIGS. 4B-4C are sequence alignments of the indicated portions of the indicated PKC isozymes, aligned using the software CLUSTAL W. In FIG. 4B, identical and homologous sequences are indicated by · and , respectively.

FIG. 4D is an alignment of a portions of several εPKC species, human, rabbit, rat, and mouse, with an indication of the V2 domain.

FIG. 5A illustrates a treatment regimen used in a study to evaluate cardiac damage in a rat heart using an ex vivo model of ischemia-reperfusion.

FIG. 5B is a graph of CPK levels in buffer perfusate determined under different conditions in the ex vivo model of ischemia-reperfusion of FIG. 5A.

FIG. 5C shows SDS-PAGE gel immunoblots from isolated mitochondria prepared from the rat hearts in the study of FIG. 5A, where the isolated mitochondria and plasma membrane fractions were probed with antibodies specific for PKCε, PKCδ, ALDH2 (used as a mitochondrial marker) and Na/K_{ATP} (used as a plasma membrane marker), the hearts having been subjected to normoxia (left lanes), IR in the absence of ψεHSP90 peptide (middle lanes), and IR in the presence of ψεHSP90 peptide (right lanes).

FIG. 5D shows SDS-PAGE gel immunoblots from co-immunoprecipitation experiments performed on isolated mitochondria from the rat hearts in the study of FIG. 5A using antibodies specific for HSP90 and PKCε for immunoprecipitation (IP) followed by the indicated antibodies for immunoblot (western blot (WB)) analysis, after ischemia and reperfusion (IR) in the absence and presence of ψεHSP90 peptide and GA.

FIGS. 6A-6B are SDS-PAGE gel immunoblots of an in vitro study using isolated rat cardiac mitochondria to determine the activation conditions required for mitochondrial translocation of εPKC. Isolated mitochondria were incubated with recombinant human εPKC in the presence of diacylglycerol/phosphatidylserine (DAG/PS), hydrogen peroxide (H₂O₂) as indicated, and in the absence of ψεHSP90 peptide (FIG. 6A) and presence of ψεHSP90 peptide (FIG. 6B). Mitochondrial PKCε levels were determined by western blotting.

FIG. 7 illustrates a mitochondrion in a cell and indicates polypeptides and other signaling molecules involved in mediating mitochondrial translocation of εPKC.

### BRIEF DESCRIPTION OF THE SEQUENCES

SEQ ID NO: 1 represents the εPKC-derived sequence, PKDNEER (amino acids 139-145) from the second variable region (V2 domain) of εPKC. This sequence is also referred to herein as ψεHSP90 peptide.

SEQ ID NO: 2 represents a HSP90-derived sequence PEDEEEK,

SEQ ID NO: 3 represents εPKC from Mus musculus; gi:6755084; ACCESSION: NP_{—}035234 XP_{—}994572 XP_{—}994601 XP_{—}994628.

SEQ ID NO: 4 represents εPKC from Rattus norvegicus; ACCESSION: NP_{—}058867 XP_{—}343013.

SEQ ID NO: 5 represents εPKC from Homo sapiens; ACCESSION: NP_{—}005391.

SEQ ID NOs: 6B71 represent variants of the εPKC-derived sequence, PKDNEER that include single or double conservative amino acid substitutions.

SEQ ID NO: 72 is the Drosophila Antennapedia homeodomain-derived carrier peptide, RQIKIWFQNRRMKVVKK.

SEQ ID NO: 73 is a carrier peptide sequence from the Transactivating Regulatory Protein (TAT, amino acids 47-57 of TAT) from the Human Immunodeficiency Virus, Type 1, YGRKKRRQRRR.

SEQ ID NO: 74 represents the conserved V2 domain of murine, rat, and human εPKC.

SEQ ID NO: 75 is a sequence from the PKCα isozyme.

SEQ ID NO: 76 is a sequence from the PKCβ isozyme.

SEQ ID NO: 77 is a sequence from the PKCγ isozyme.

SEQ ID NO: 78 is a sequence from the PKCθ isozyme.

SEQ ID NO: 79 is a sequence from the PKCδ isozyme.

SEQ ID NO: 80 is a sequence from the PKCε isozyme.

SEQ ID NO: 81 is a sequence from the PKCη isozyme.

SEQ ID NO: 82 is a sequence from the PKCβI isozyme.

SEQ ID NO: 83 is a sequence from the PKCβII isozyme.

SEQ ID NO: 84 is a sequence from the PKCα isozyme,

SEQ ID NO: 85 is a sequence from the PKCγ isozyme.

SEQ ID NO: 86 is a sequence from the PKCδ isozyme.

SEQ ID NO: 87 is a sequence from the PKCθ isozyme.

SEQ ID NO: 88 is a sequence from the PKCε isozyme.

SEQ ID NO: 89 is a sequence from the PKC-eta isozyme.

SEQ ID NO: 90 is a sequence from the PKC-zeta isozyme,

SEQ ID NO: 91 is a portion of the human PKCε isozyme.

SEQ ID NO: 92 is a portion of the rabbit PKCε isozyme,

SEQ ID NO: 93 is a portion of the rat PKCε isozyme.

SEQ ID NO: 94 is a portion of the mouse PKCε isozyme.

SEQ ID NO: 95 corresponds to amino acid residues 130-153 of the sequences identified as SEQ ID NO: 91, SEQ ID NO: 92, SEQ ID NO: 93, and SEQ ID NO: 94, and is referred to herein as the V2 domain of epsilon PKC.

### DETAILED DESCRIPTION

### I. Definitions

Prior to describing the present compositions and methods, the following terms are defined for clarity,

As used herein a “conserved set” of amino acids refers to a contiguous sequence of amino acids that is identical or closely homologous (e.g., having only conservative amino acid substitutions) between members of a group of proteins. A conserved set may vary in length, and can be anywhere from five to over 50 amino acid residues in length, or can be between 5-25, 5-20, 5-15, 5-12, 6-15, 6-14, 6-12, 8-20, 8-15, or 8-12 residues in length.

As used herein, a “conservative amino acid substitutions” are substitutions that do not result in a significant change in the activity or tertiary structure of a selected polypeptide or protein. Such substitutions typically involve replacing a selected amino acid residue with a different residue having similar physico-chemical properties. For example, substitution of Glu for Asp is considered a conservative substitution since both are similarly-sized negatively-charged amino acids. Groupings of amino acids by physico-chemical properties are known to those of skill in the art and available in most basic biochemistry texts.

As used herein, the terms “domain” and “region” are used interchangeably to refer to a contiguous sequence of amino acids within a protein characterized by possessing a particular structural feature or function, such as a helix, sheet, loop, binding determinant for a substrate, enzymatic activity, signal sequence and the like.

As used herein, the terms “peptide” and “polypeptide” are used interchangeably to refer to a compound made up of a chain of amino acid residues linked by peptide bonds. Unless otherwise indicated, the sequence for peptides is given in the order from the “N” (or amino) terminus to the “C” (or carboxyl) terminus.

Two amino acid sequences or two nucleotide sequences are considered “homologous” (as this term is preferably used in this specification) if they have an alignment score of >5 (in standard deviation units) using the program ALIGN with the mutation gap matrix and a gap penalty of 6 or greater (Dayhoff, M. O., in Atlas of Protein Sequence and Structure (1972) Vol. 5, National Biomedical Research Foundation, pp. 101-110, and Supplement 2 to this volume, pp 1-10.) The two sequences (or parts thereof) are more preferably homologous if their amino acids are greater than or equal to 50%, more preferably 70%, more preferably 80%, 85%, 90%, 91%, 92%, 93%, 94%, 95%, 96%, 97%, 98% or 99% identical when optimally aligned using the ALIGN program mentioned above. In other embodiments, sequences are homologous if their amino acids are 80-95%, 85-95%, 95-100% identical, inclusive of the ranges. In further embodiments, the sequences are homologous if their amino acids are 85, 86, 87, 88, 89, 90, 91, 92, 93, 94, 95, 96, 97, 98, 99, or 100% identical.

A peptide or peptide fragment is “derived from” a parent peptide or polypeptide if it has an amino acid sequence that is homologous to the amino acid sequence of, or is a conserved fragment from, the parent peptide or polypeptide.

The term “effective amount” means a dosage sufficient to provide treatment for the disorder or disease state being treated. This will vary depending on the patient, the disease and the treatment being effected.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

As used herein, “modulating εPKC and HSP90 interactions” means increasing or decreasing intermolecular interactions between εPKC and HSP90. In some embodiments, the intermolecular interactions are increased, thereby promoting ischemia/reperfusion-associated cytoprotection.

As used herein, “modulating translocation of εPKC to the mitochondria” means increasing or decreasing translocation of εPKC from the cytoplasm to the mitochondria. In embodiments, the peptide is translocated to the mitochondrial membrane (outer and/or inner), and/or interior compartments (such as the matrix).

Abbreviations for amino acid residues are the standard 3-letter and/or 1-letter codes used in the art to refer to one of the 20 common L-amino acids.

As used herein and in the appended claims, the singular forms “a”, “an”, and “the” include plural reference, unless the context clearly dictates otherwise.

Unless defined otherwise, all technical and scientific terms used herein have the same meanings as commonly understood by one of ordinary skill in the art to which this subject matter belongs. Although any methods and materials similar or equivalent to those described herein can be used in the practice or testing of the present subject matter, the preferred methods, devices, and materials are now described.

All publications mentioned herein are incorporated herein by reference for the purpose of describing and disclosing the methodologies which are reported in the publications which might be used in connection with the subject matter herein.

Protein sequences are presented herein using the one letter or three letter amino acid symbols as commonly used in the art and in accordance with the recommendations of the IUPAC-IUB Biochemical Nomenclature Commission.

### II. Therapeutic Peptides

In one aspect, peptides effective to modulate translocation of εPKC (epsilon PKC) to the mitochondria are described. In another aspect, peptides effective to modulate interactions between εPKC and HSP90 are described. In yet another aspect, peptides effective to modulate mitochondrial import of a cytosolic polypeptide are described.

Previous studies have shown that the protein epsilon protein kinase C (εPKC) is involved in the endogenous signaling pathway that protects the myocardium during reperfusion following ischemia (Chen, L., et al., Proc Natl Acad Sci USA, 2001, 98:11114-9). Targets of εPKC are known to reside in the cardiac mitochondria. However, εPKC is a cytosolic protein, and it was heretofore unknown how εPKC was imported into mitochondria, and the identity and function of its cellular and/or mitochondrial binding partners in the mitochondria.

Heat shock protein 90 (HSP90) is a chaperone protein that prevents the misfolding of cellular proteins in response to cellular stress (Pearl, L. H. and C, Prodromou, Annu Rev Biochem, 2006. 75:271-94). Ischemia and reperfusion (IR), as occur(s) in diseases and conditions such as myocardial infarction and other injures to the heart, are known to cause increased oxidation and misfolding of cellular proteins (Latchman, D. S., Cardiovasc Res, 2001. 51:637-46). HSP90 also appears to play a role in mitochondrial import of cytosolic proteins (Young, J. C., N. J, Hoogenraad, and F. U. Hartl, Cell, 2003. 112:41-50).

Based, in part, on experiments and observations described, herein, it has been discovered that HSP90 mediates the IR-induced mitochondrial translocation of εPKC. Interfering with HSP90 function reduces the cytoprotection afforded by εPKC translocation to the mitochondria during the early stage of reperfusion. It has further been discovered that a peptide representing a region of homology between εPKC and HSP90 modulates εPKC—mediated cytoprotection, apparently by stabilizing εPKC in a conformation that increases its interaction with HSP90. The studies leading to this discovery and the peptides identified are now described.

A. Cytoprotection by HSP90 During Reperfusion

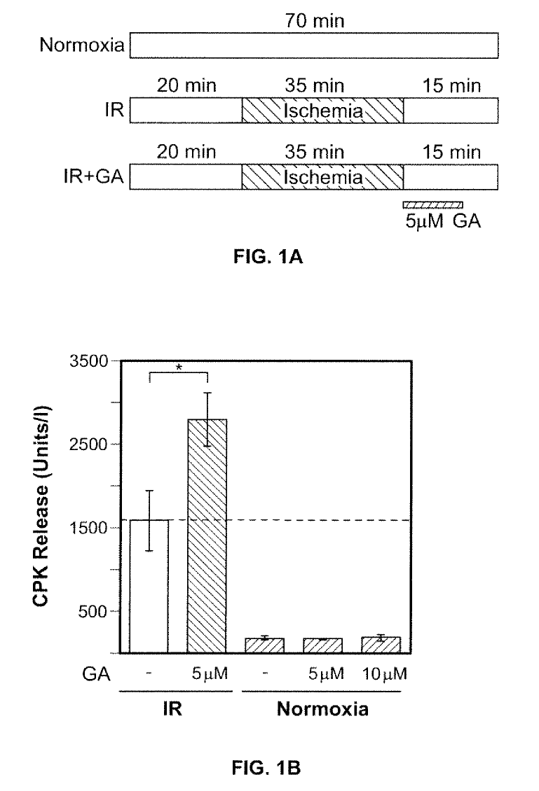

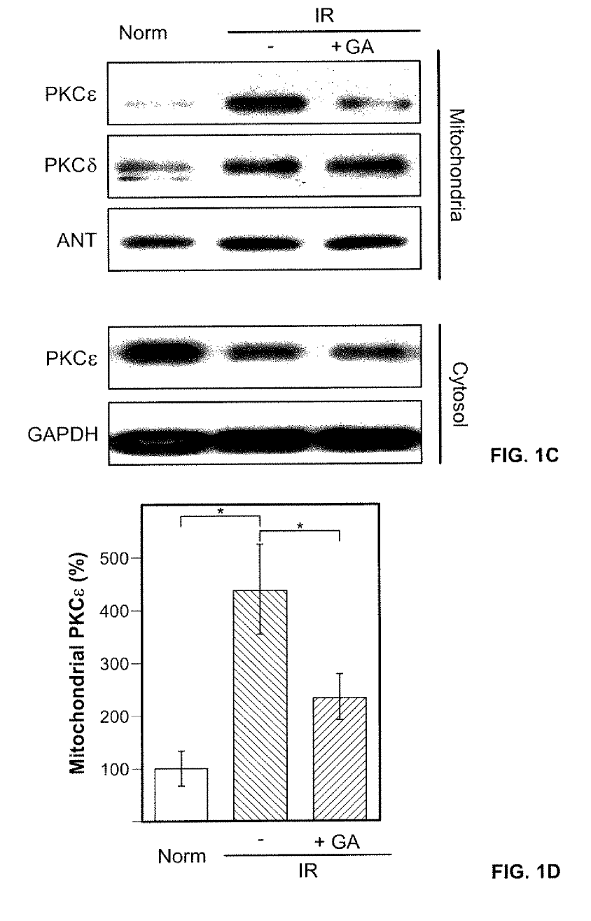

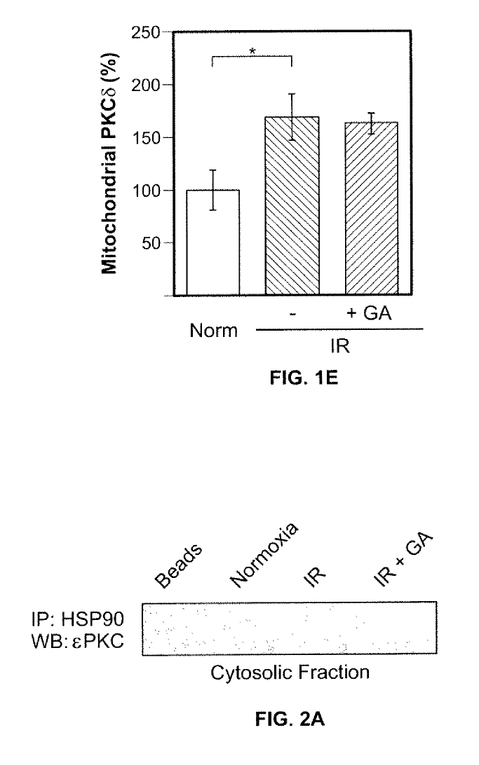

In accordance with a well-established ex vivo rat heart model for ischemia-reperfusion (see, e.g., U.S. Publication Nos. 20080167247, 20080153926, 20070299012, and 20060293237, which are herein incorporated by reference) and the method described in Example 1, hearts were removed from animals (rats) and subjected to a normoxia or an IR protocol, both depicted in FIG. 1A. The normoxia regimen comprised exposing hearts to 70 minutes of a normal oxygen state. The IR (ischemia/reperfusion) regimens comprised subjecting hearts to 20 minutes of normoxia, followed by 35 minutes of ischemia, followed by 15 minutes of reperfusion. The IR+GA regimen was similar to IR regimen, expect with the addition of 5 μM geldanamycin (GA), an HSP90-selective inhibitor (Whitesell L. and Cook P. Mol. Endocrinol 1996. 10: 705712), present during a portion of the reperfusion period. The amount of necrotic cell death was then determined by measuring the release of creatine phosphokinase (CPK) into the buffer perfusate during reperfusion, which served as an indicator of ischemic damage. The results are shown in FIG. 1B.

Subjecting the perfused hearts to IR resulted in a significant increase in CPK release compared to control, as seen in FIG. 1B. Inhibition of HSP90 with GA further increased injury, as evidenced by a 176% increase in CPK release (n=7; p<0.05). Notably, inhibiting HSP90 with 5 μm or even 10 μm GA had no effect on CPK release in the absence of IR injury, ruling out a direct effect of GA under these conditions.

This data suggested that HSP90 activity contributed to the endogenous cytoprotective effect observed during the initial stages of reperfusion of ischemic myocardium.

B. Ischemia-Reperfusion-induced Mitochondrial Translocation of εPKC

To investigate the relationship between εPKC and HSP90 in cytoprotection during ischemia and reperfusion, the effects of HSP90 inhibition on εPKC translocation to cardiac mitochondria was examined. As shown in FIG. 1C, IR induced the translocation of both the εPKC and δPKC isozymes to mitochondria (438% and 169%, respectively). However, inhibition of HSP90 with GA (5 μm) during reperfusion significantly attenuated the IR-induced mitochondrial translocation of εPKC, as shown by comparing the middle and right lanes in the uppermost immunoblot of FIG. 1C and in the corresponding histogram of FIG. 1D, but had no effect on IR-induced mitochondrial translocation of δPKC, as shown by comparing the middle and right lanes in the “PKCδ” immunoblot of FIG. 1C and in the corresponding histogram of FIG. 1E, suggesting that HSP90 specifically regulates εPKC translocation.

These results are consistent with the opposing roles of εPKC and δPKC in regulating the response of the myocardium to IR injury (Budas, et al., Pharmacol Res, 2007. 55:523-36). Activation of εPKC is cardioprotective, whereas activation of δPKC worsens injury (Chen, L., et al., Proc Natl Acad Sci USA, 2001. 98:11114-9; Budas, G. R, E. N. Churchill, and D. Mochly-Rosen, Pharmacol Res, 2007. 55:523-3; Murriel, C. L., et al, J Biol Chem, 2004. 279:47985-91; and Chen, C. and D. Mochly-Rosen, J Mol Cell Cardiol, 2001. 33:581-5). In particular, εPKC activation inhibits MPTP (Baines, C. P., et a., Circ Res, 2003. 92:873-80), opens mitoK_{ATP} channels (Jaburek, M., et al., Circ Res, 2006. 99:878-83) increases the activity of COIV (Ogbi, M., et al., Biochem J, 2004. 382:923-32) and ALDH2 (Chen C. H. et al., Science 2008. 321:1493-1495), resulting in cytoprotection, while mitochondrial translocation of δPKC triggers necrotic and apoptoic cell death pathways (Murriel, C. L., et al., J Biol Chem, 2004. 279:47985-91) by reducing ATP regeneration through inhibition of PDH (Churchill, E. N., et al., Circ Res, 2005. 97: p. 78-85), increasing ROS generation, and increasing cytochrome c release by increasing the Bad/Bcl-2 ratio (Churchill, E. N., et al., Circ Res, 2005. 97: p. 78-85).

In view of the opposing roles of εPKC and δPKC, selectively blocking mitochondrial translocation of εPKC by inhibiting HSP90 simultaneously prevents the cytoprotective effects of εPKC and increases δPKC-mediated cell death, exacerbating the damage to the cells and tissues.

C. Inhibiting HSP90 Prevents IR-Induced Association of εPKC and HSP90

Having established that IR-induced translocation of εPKC is modulated or abolished by HSP90 inhibition, studies were performed to determine whether HSP90 and εPKC physically associate, and in which subcellular compartment association may occur. To this end, a co-immunoprecipitation strategy was employed in which different mitochondrial preparations were subjected to immunoprecipitation using a first antibody, and immunoblotting was subsequently performed on the precipitated material using a second antibody to determine if co-immunoprecipitation had occurred (Example 2). Since εPKC and HSP90 are predominately cytosolic proteins, it was expected to observe co-immunoprecipitation of εPKC and HSP90 in the cytosolic fraction following exposure to IR.

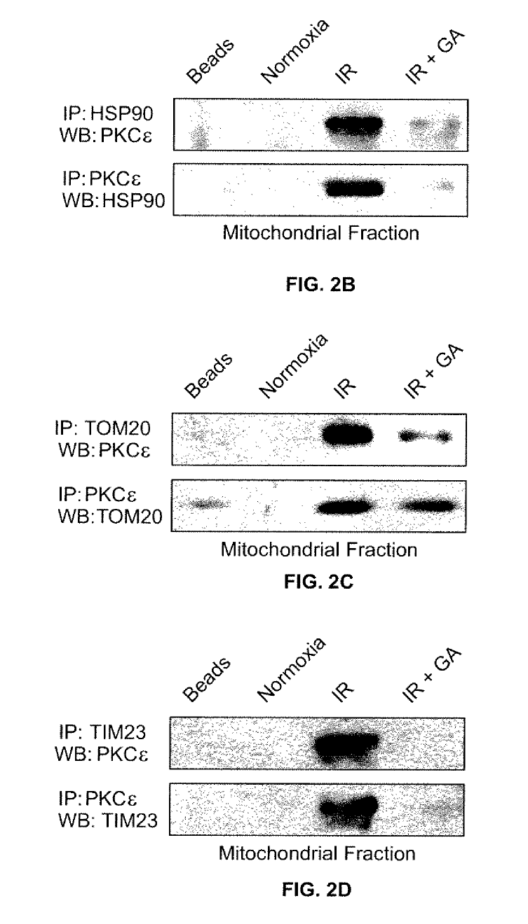

Surprisingly, no association was observed between HSP90 and εPKC in the cytosol under any conditions tested, as seen in FIG. 2A, and detailed in Example 2. Further, no association between HSP90 and εPKC was observed under basal (i.e., non-ischemic) conditions in the mitochondrial fraction (FIG. 2B, normoxia condition). However, when hearts were subjected to IR, εPKC co-immunoprecipitated with HSP90 in the mitochondrial fraction and this association was blocked by treatment with 5 μm GA, as seen in FIG. 2B (upper panel). This association was confirmed by reverse immunoprecipitation (FIG. 2B, lower panel).

As noted above, εPKC has been shown to associate with several different intra-mitochondrial substrates (Baines, C. P., et al., Circ Res, 2003. 92:873-80; Ping, P., et al., Circ Res, 2001. 88:59-62; Jaburek, M., et al., Circ Res, 2006. 99:878-83; and Ogbi, M., et al., Biochem J, 2004. 382:923-32); however the mechanism by which εPKC enters the mitochondria has not been determined. Import of mitochondrial proteins is known to be mediated by import machinery including the translocase of the outer mitochondrial membrane (“Tom”), a multi-protein complex that consists of the receptor subunits Tom20, Tom70 and Tom 22 and the membrane-embedded subunits Tom40, Tom7, Tom6 and Tom5. This complex in conjunction with the translocase of the inner mitochondrial membrane (“Tim”) mediates the import of cytosolic proteins into the mitochondria across the outer mitochondrial membrane (OMM). Recent studies have suggested that HSP90-chaperoned proteins enter cardiac mitochondria via interaction with the Tom20 subunit and that Tom20 is critical for protection from IR injury (Boengler, K., et al., J Mol Cell Cardiol, 2006. 41:426-30).

As shown, substantial co-immunoprecipitation of Tom20 and εPKC was observed following exposure to IR, which was abolished by 5 μm GA (FIG. 2C; n=3) A similar HSP90-dependent interaction of εPKC with Tim23 was observed following IR; i.e., association of PKC with Tim23 substantially decreased following IR when hearts were treated with GA (FIG. 2D,; n=3). These results suggest that εPKC interacts with components of the mitochondrial import machinery in an HSP90-dependent manner following IR.

These studies demonstrate that εPKC and HSP90 do not form a complex under basal (non-IR) conditions, but that they physically associate on cardiac mitochondria following the stimulus of ischemia and reperfusion. Interaction of HSP90 with εPKC was not observed under unstimulated (normoxic) conditions and the IR-induced interaction was substantially reduced when HSP90 was inhibited with geldanamycin. Furthermore, an HSP90/εPKC complex was not found in the cytosolic fraction, where these proteins are also present, suggesting stimulus-induced association between HSP90 and εPKC at the mitochondria in response to IR.

D. Association of εPKC with the Matrix Side of the IMM Following IR

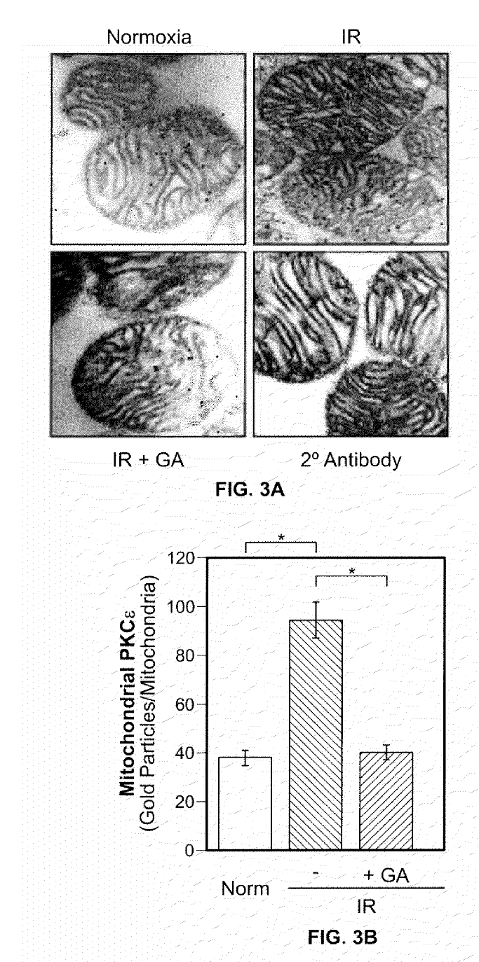

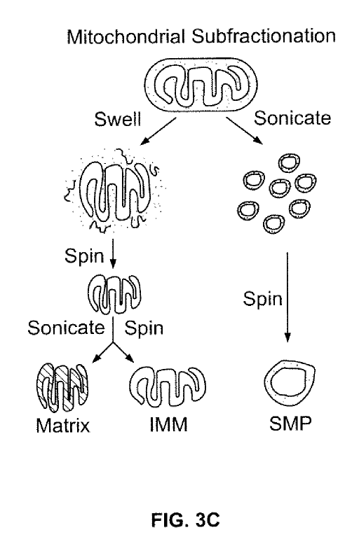

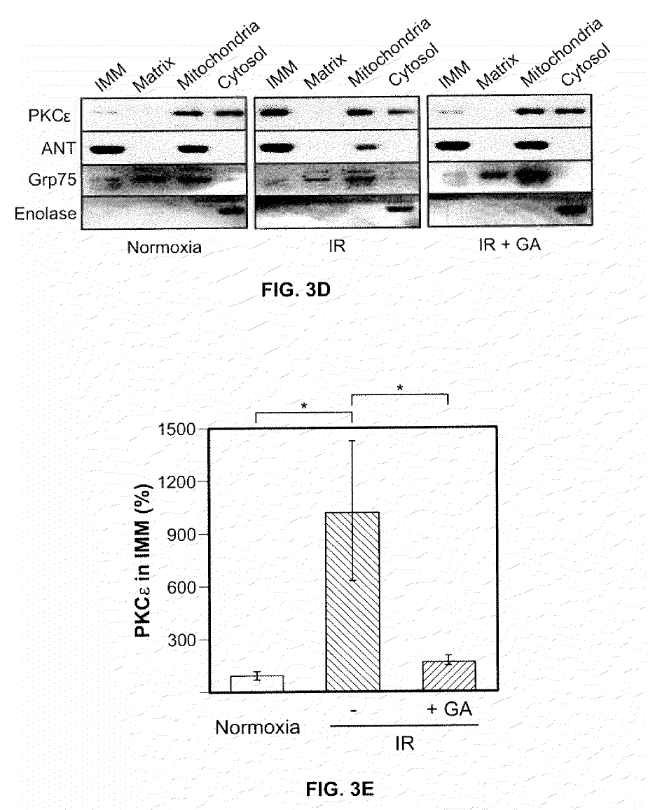

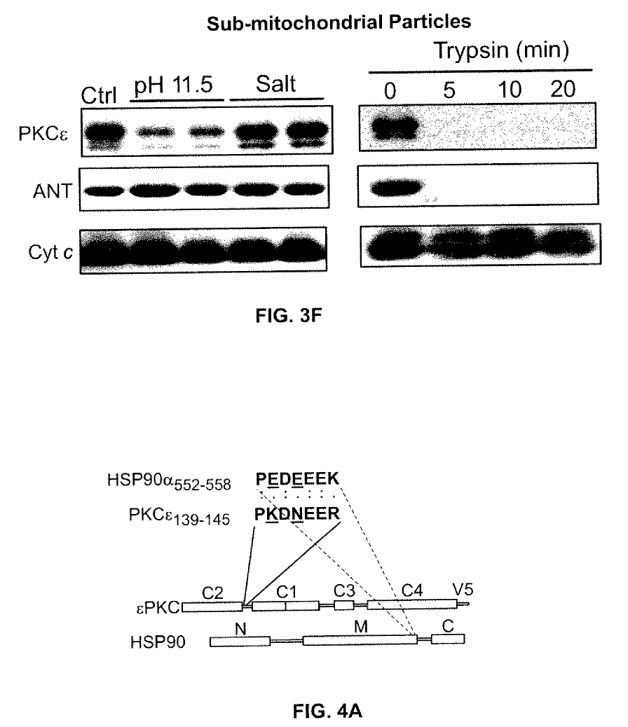

Having observed IR-induced and HSP90-dependent mitochondrial translocation and association of εPKC with the mitochondrial import machinery, immunogold electron microscopy using an εPKC-specific antibody was performed with isolated mitochondria and mitochondrial subfractions, as detailed in Example 3 and shown in FIGS. 3A-3F.

Under normoxic conditions εPKC was found residing within cardiac mitochondria as evidenced by immunogold staining for εPKC by electron microscopy (FIG. 3A, upper left panel). However, following IR a 250% increase in the amount of εPKC immunogold labeling within mitochondria was observed. εPKC was present predominately at or near the inner mitochondrial membrane (FIG. 3A, upper right panel). The IR-induced increase in mitochondrial εPKC (FIG. 3A, upper right panel) was prevented when HSP90 was inhibited by GA (FIG. 3B, lower left panel). There was a complete absence of immunolabelling when mitochondria were incubated with the immunogold-conjugated secondary antibody alone (i.e. in the absence of the εPKC antibody (FIG. 3A, lower right panel) ruling out any non-specific binding of the gold-conjugated secondary antibody.

To further investigate the association of εPKC with the inner mitochondrial membrane, mitochondria were fractionated to obtain matrix, inner mitochondrial membrane (IMM) and sub-mitochondrial particle (SMP) fractions (FIG. 3C) by standard methods (Pagliarini, D. J., et al, Mol Cell, 2005. 19:197-207). Several known antibodies were used to confirm the correct fractionation of the mitochondria. Analysis of the fractionation is shown in FIGS. 3D-3E, and reveals that IR increased εPKC association with the IMM fraction and that this interaction was abolished by inhibiting HSP90 with GA, in confirmation of the electron microscopic analysis.

To confirm that PKCε can associate with the IMM, sub-mitochondrial particles (SMPs) were prepared from hearts subjected to IR, SMP vesicles were orientated “inside out”, exposing IMM-associated proteins that face the matrix while sequestering proteins that face the inner mitochondrial space within the inverted mitochondrial vesicle (as shown in FIG. 3C). Exposure to carbonate wash at pH 11.5 (used to remove strongly associated, membrane-associated proteins) removed εPKC from the IMM, whereas exposure to 400 mM KCl high-salt wash (used to remove loosely associated proteins) did not (FIG. 3F, upper left panel). These findings suggest a tight interaction between εPKC and the IMM. Trypsin, which cannot cross membranes, completely removed εPKC from these inside-out mitochondrial vesicles (FIG. 3F upper right panel). That trypsin could access εPKC suggests that εPKC is present on the matrix side of the IMM, which is exposed to trypsin in the SMP preparation. In contrast, levels of cytochrome c, which in the inner membrane space between the inner and outer mitochondrial membranes (and therefore resides inside the SMP vesicles), were unchanged by trypsin digestion (FIG. 3F, lower left and right panels).

The results of the fractionation experiments confirm the results of immunoblot analysis and electron microscopic analysis, further demonstrating that εPKC was present inside cardiac mitochondria, and that intra-mitochondrial εPKC levels were increased by IR, in an HSP90-dependent manner.

### E. Identification of Peptide Sequences

The observations above indicated that εPKC and HSP90 physically associate in the mitochondria in a stimulus and HSP90-dependent manner. Peptides capable of modulating this interaction were sought.

It was previously found that the primary sequence of the εPKC binding protein, εRACK, shares a short sequence of homology with the C2 domain εPKC (Dorn, G. W. et al., Proc Natl Acad Sci USA, 1999. 96:12798-803) and that an eight amino acid peptide derived from this region of homology (termed ψεRACK) was an allosteric agonist of εPKC. The peptide interfered with the auto-inhibitory intramolecular interaction between the ψεRACK site and the εRACK-binding site in εPKC, thereby stabilizing a conformational state in which the εRACK-binding site on εPKC was available for protein-protein interaction. Thus, the ψεRACK peptide enhanced the binding of εPKC to εRACK and promoted εPKC translocation and activation (Dorn, G. W. et al, Proc Natl Acad Sci USA, 1999. 96:12798-803).

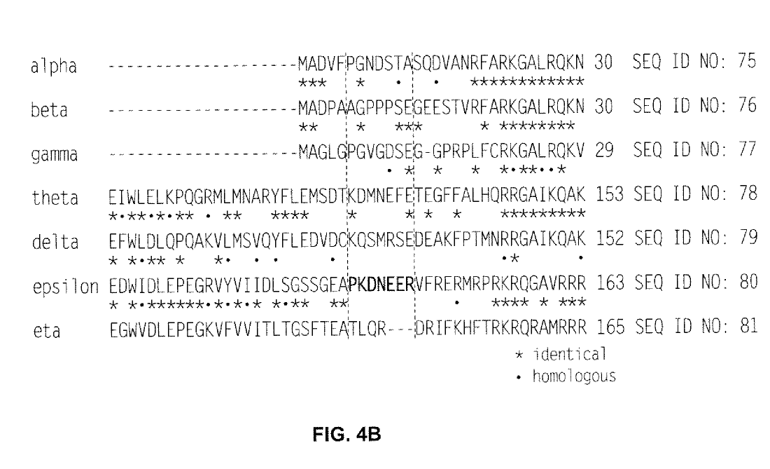

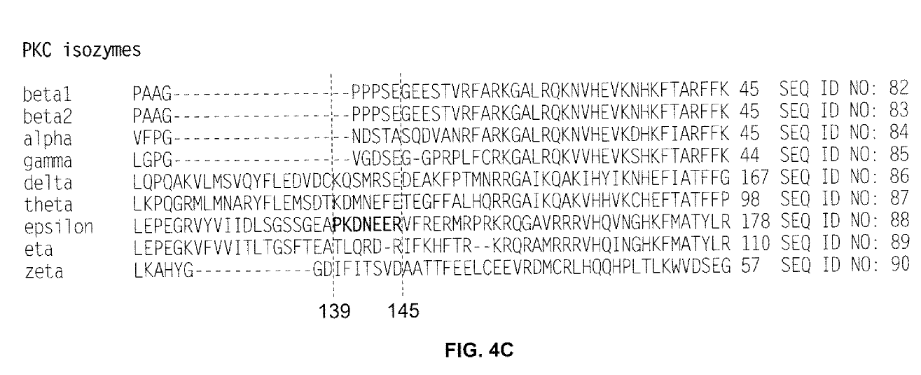

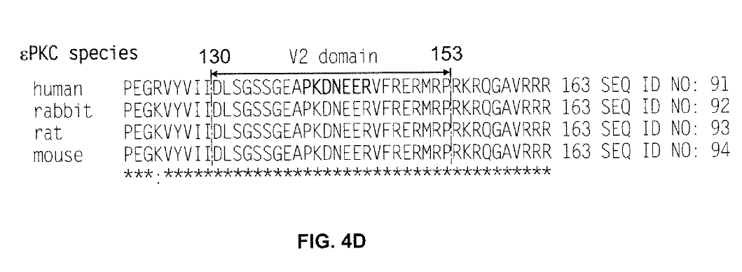

Using LALIGN software a region of homology between εPKC and HSP90 was identified. This εPKC-derived sequence, PKDNEER (amino acids 139-145; SEQ ID NO: 1), designated ψεHSP90, resided in the second variable region or V2 domain of εPKC. The V2 domain ranges from amino acid 130 to amino acid 153 on εPKC and resides between the C1 domain and the C2 domain of εPKC, as depicted in FIG. 4A. This sequence is homologous to PEDEEEK, found at the middle-terminal domain of HSP90 (amino acids 552-558 on HSP90α and 544-550 on HSP90β) located on the middle domain of HSP90, which is involved in binding to HSP90-chaperoned proteins. There is a charge difference between these homologous peptides (Lys¹⁴⁰ and Asn¹⁴² on PKCε compared with Glu⁵⁵³ and Glu⁵⁵⁵ on HSP90α; underlined in FIG. 4A).

The HSP90-homologous sequence in εPKC is unique in that it is not found in any other members of the PKC family, as evident from the alignments shown in FIGS. 4B-4C. The sequence alignments in FIGS. 4B-4C were done using CLUSTAL W software (Thompson J. D. et al., Nucl Acids Res. 1994 22: 4673-4680). The sequence is also evolutionary conserved among εPKCs from different species, as seen in the alignments of partial sequences from mouse εPKC (SEQ ID NO: 3), rat εPKC (SEQ ID NO: 4), human εPKC (SEQ ID NO: 5) and rabbit εPKC in FIG. 4D.

Accordingly, in one embodiment, an isolated peptide that consists of a sequence of amino acid residues selected from a contiguous sequence of amino acid residues from the V2 domain of εPKC is provided. In various embodiments, the isolated peptide consists of from between about 3-15, 3-12, 3-8, 3-7,3-6, 3-5, 4-24, 4-15, 4-12, 4-8, 4-7, 4-6, 4-5, 5-24, 5-15, 5-12, 5-10, 5-8, 5-7, 5-6, 6-24, 6-15, 6-12, 6-10, 6-8, 6-7, 7-24, 7-15, 7-12, 7-10, 7-8, 8-24,8-15, 8-12, 8-10, or 8-9 contiguous amino acid residues from the V2 domain of εPKC. These ranges are contemplated as inclusive. For example, where the range is stated as 6-8 amino acids, ranges of 6-7 and 7-8 are contemplated. In preferred embodiments, the isolated peptide consists of a sequence of amino acid residues from a V2 domain of εPKC identified as SEQ ID NO, 91, SEQ ID NO: 92, SEQ ID NO: 93, and SEQ ID NO: 94. In another preferred embodiment, the peptide consists of a sequence of amino acid residues from residues 130-153, inclusive, of the sequences identified as SEQ ID NO: 91, SEQ ID NO: 92, SEQ ID NO: 93, and SEQ ID NO: 94 (SEQ ID NOs: 95-98). Excluded herein are any peptide sequences identical to the βPKC V2 peptides as disclosed in U.S. Pat. No. 5,783,405.

F. Ex vivo Delivery of Peptides to Whole Hearts

Having identified a region of primary sequence homology between εPKC and HSP90, it was unknown whether the peptides derived from this region would modulate the association of εPKC and HSP90, or whether they would affect the response of the myocardium to IR. It could not be predicted a priori whether such a peptide would act as a competitive inhibitor (i.e., blocking inter-molecular interactions), act as an allosteric agonist (i.e., by interfering with intra-molecular interactions), act in a different manner, or have any affect at all.

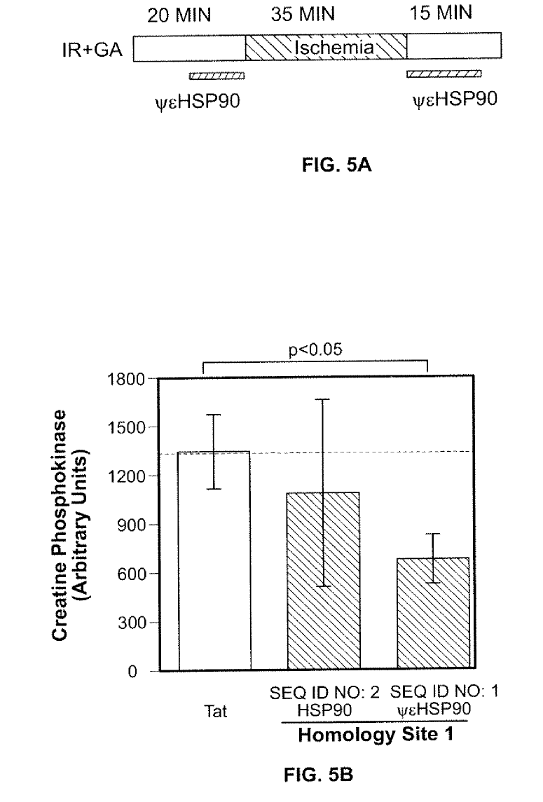

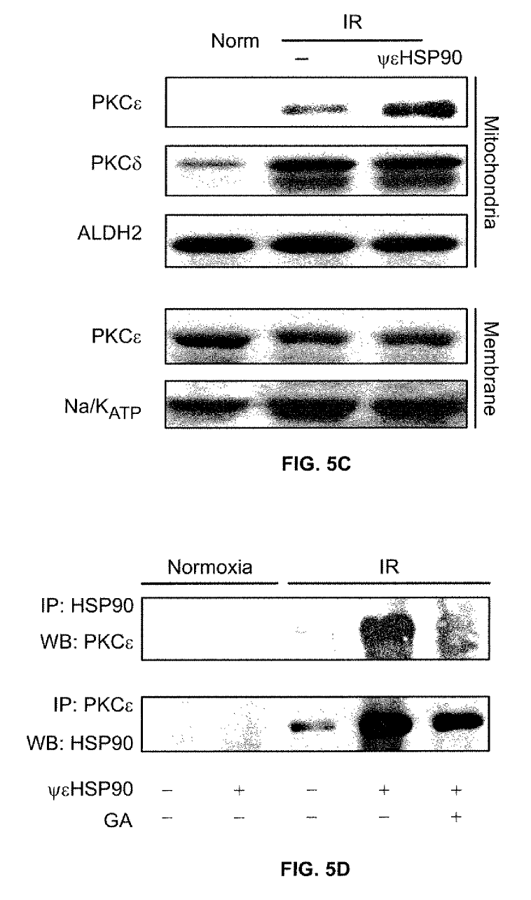

Both the εPKC-derived peptide (i.e., PKDNEER; SEQ ID NO. 1) and the HSP90-derived peptide (i.e., PEDEEEK; SEQ ID NO: 2) were tested for their activities in a first study using a rat ex vivo model of IR. In this study, detailed in Example 4, the peptides were rendered cell permeable by conjugation to a TAT protein-derived carrier peptide (TAT₄₇₋₅₇) via a cysteine-cysteine bond at their N-termini (Chen, L., et al., Proc Natl Acad Sci USA, 2001. 98:11114-9). Hearts removed from animals were subjected to IR according to the protocol in FIG. 5A, where each of the Tat-conjugated peptides was administered 10 minutes before ischemia and for the first 10 minutes of reperfusion, as depicted in FIG. 5A. The levels of CPK released into the cardiac perfusate was quantified, and as seen in FIG. 5B, were reduced by 47% in hearts that were treated with the εPKC-derived ψεHSP90 peptide (SEQ ID NO: 1), while the peptide derived from HSP90 (SEQ ID NO: 2) had no statistical effect on IR-induced CPK release.

It was also determined whether treatment with ψεHSP90 peptide (SEQ ID NO: 1) increased mitochondrial translocation of PKCε in vivo. As described in Example 4, isolated mitochondria from hearts subjected to IR in the presence of 1 μm ψεHSP90 peptide were immunoblotted for detection of εPKC and δPKC. As seen in FIG. 5C, treatment with the ψεHSP90 peptide induced ˜20% higher PKCε levels (right lanes) when compared to the mitochondria from the IR group untreated with peptide (middle lanes). PKCδ association with the mitochondria after IR was not altered by ψεHSP90 treatment (immunoblot labeled “PKCδ” in FIG. 5C). Furthermore, ψεHSP90 did not affect PKCε translocation to the plasma membrane (FIG. 5C, lower panels). As seen in FIG. 5D, ψεHSP90 treatment resulted in a ˜4 fold increase in IR-induced physical interaction between HSP90 and PKCε, as detected by co-immunoprecipitation (“IP”, “WB”=Western Blot) which was greatly attenuated by GA.

These results demonstrated that the εPKC V2 domain-derived ψεHSP90 peptide modulated the interaction between εPKC and HSP90, thereby affecting cytoprotection associated with reperfusion.

G. Mitochondrial Translocation of PKCε In Vitro

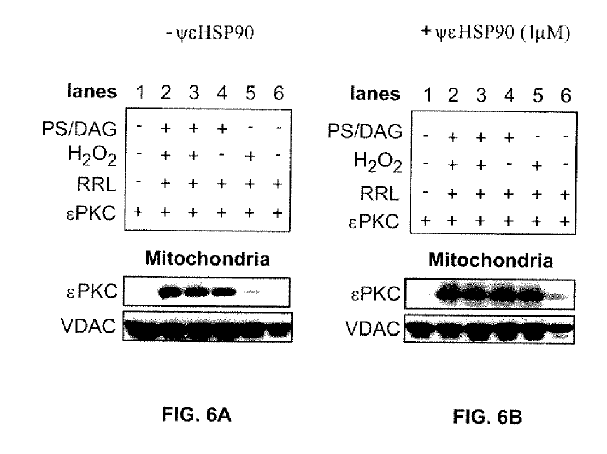

An in vitro approach was used to identify cellular components that were important for εPKC translocation to the mitochondria. As described in Example 5, mitochondria were isolated from normoxic hearts and incubated for 20 minutes at 37° C. with purified recombinant GST-tagged εPKC (Cell Signaling Technology Inc,), which was pretreated by different combinations of PKC activation components including phospholipids and hydrogen peroxide (H₂O₂) in the absence (FIG. 6A) and presence (FIG. 6B) of the ψεHSP90 peptide. Following incubation of PKCε with its activation components, mitochondria were introduced into the εPKC mixture and incubated for an additional 20 minutes, after which the mitochondria were pelleted by centrifugation and then probed with antibodies selective for PKCε and the mitochondrial marker VDAC The use of GST-tagged εPKC allowed the distinction, based on size, between exogenous GST-εPKC and native/endogenous εPKC present in the mitochondria prior to treatment. Rabbit reticulocyte lysate (RRL) was used as an exogenous source of HSP90 as described previously (Scherrer et al., Biochemistry. 1992. 31:7325-7329)

εPKC is known to require phosphatidylserine (PS) and diacylglycerol (DAG) but not Ca²⁺ for translocation. Consistent with previous observations, it was found that diacylglycerol was required to induce mitochondrial translocation of εPKC, as seen in FIG. 6A (lanes 2, 3, 4) and that the absence of diacylglycerol precluded the translocation of εPKC (FIG. 6A, lanes 1, 5, 6). The ψεHSP90 peptide increased the association of εPKC with the mitochondria (FIG. 6B, lanes 2, 3, 4, 5). Furthermore, treatment with the ψεHSP90 peptide resulted in translocation of εPKC in the presence of hydrogen peroxide despite the absence of phospholipids, suggesting that ψεHSP90 acted in an allosteric manner, inducing the mitochondrial translocation of εPKC in the absence of phospholipid stimulation (FIG. 6B, lane 5).

H. Interaction of εPKC and HSP90

The results described herein suggest that the molecular chaperone HSP90 is necessary for the mitochondrial translocation and import of εPKC and that the interaction between εPKC and HSP90 is important for increasing cell viability during the early stages of reperfusion following myocardial ischemia. Inhibiting HSP90 abolished IR-induced translocation of εPKC, decreasing cytoprotection against IR-induced damage.

A peptide derived from the V2 domain, and which represents a region of homology between εPKC and HSP90, modulated the interaction between εPKC and HSP90. In particular, the peptide increased the cytoprotection afforded by εPKC. Without being limited to a theory, it is believed that the εPKC-derived peptide enhanced the interaction between εPKC and HSP90, possibly by disrupting intra-molecular interactions within or between εPKC protein molecules, thereby stabilizing εPKC in a conformation suitable for interacting with HSP90.

These results provide the basis for understanding the mechanism of mitochondrial translocation and importation of εPKC and the resulting cytoprotective effects of εPKC that mitigate damage due to ischemic injury. The results also suggest that peptides derived from the V2 domain of εPKC can be used to reduce or prevent IR-induced cell damage, and possibly treat a variety of diseases and disorders that have a basis in mitochondrial dysfunction or oxidative stress.

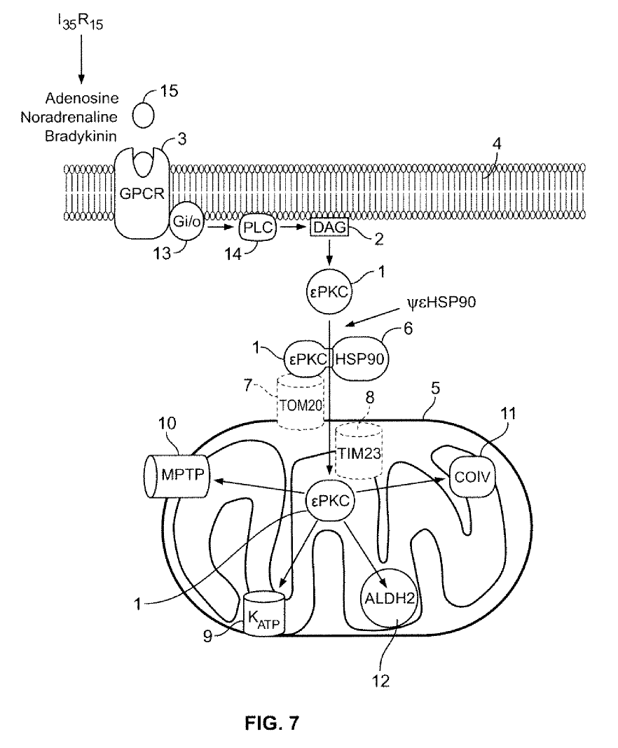

A proposed model for the import of εPKC into cardiac mitochondria is illustrated in FIG. 7. According to the model, cytosolic εPKC 1 exists in the inactive conformation until stimulation by the phospholipid-derived, second messenger diacyl glycerol (DAG) 2, which is downstream of G-protein coupled receptor (GPCR) 3 (i.e., in a plasma membrane 4) occupancy with molecules that accumulate during ischemia (such as adenosine and noradrenaline). On activation, εPKC 1 undergoes a conformational change and translocates to cardiac mitochondria 5, whereupon it forms a complex with the molecular chaperone, HSP90 6.

HSP90 6 permits mitochondrial import of εPKC 1 through translocases of the outer membrane (TOM20 7) and translocase of the inner membrane (TIM23 8) complexes, permitting εPKC 1 to reach its intra-mitochondrial cytoprotective targets such as mitochondrial ATP sensitive K⁺ channels (mitoK_{ATP} 9), the mitochondrial permeability transition pore (MPTP 10) complex IV of the electron transport chain (COIV 11) and mitochondrial aldehyde dehydrogenase 2 (ALDH2 12) which have been previously recognized to be εPKC cardioprotective targets, essential for cell viability following ischemic injury. Other plasma membrane 4 proteins such as a G-protein (Gi/o 13), phosholipase C (PLC 14), and a GPCR 3 ligand 15 are indicated.

Treatment with ψεHSP90, which mimics an intramolecular interaction site between εPKC and HSP90, results in allosteric εPKC activation and enhances translocation of εPKC to cardiac mitochondria. By permitting εPKC to reach its cytoprotective mitochondrial targets, ψεHSP90 reduces necrotic cell death induced by myocardial ischemia/reperfusion injury.

III Compositions for Modulating the Interaction between εPKC and HSP90

The ψεHSP90 peptide described herein was identified by a sequence homology search between εPKC and HSP90. The homologous sequence between the two proteins was a short stretch of seven amino acids in which four of the seven amino acids were identical. These sequences also displayed a charge difference (Lys¹⁴⁰ and Asn¹⁴² on human PKCε compared with Glu⁵⁵³ and Glu⁵⁵⁵ on human HSP90, previously found to be indicative of a protein-protein interaction for PKC. When tested in the ex vivo model of IR, the ψεHSP90 peptide, derived from εPKC significantly reduced IR injury, whereas the corresponding sequence from the HSP90 protein had no effect.

This ψεHSP90 peptide was designed to modulate specific interaction (HSP90 binding) and should affect only specific subcellular εPKC function (i.e. mitochondrial εPKC function) without altering global cellular εPKC activity. The ψεHSP90 peptide is believed to work in an allosteric manner, stabilizing εPKC in a conformation that is favorable to HSP90 binding, rather than, e.g., RACK binding.

The ψεHSP90 peptide and related peptides have therapeutic potential in the treatment of ischemic heart disease and acute oxidative-stress related diseases/conditions such as myocardial infarction, stroke, and transplantation. The ψεHSP90 peptide and related peptides may further have therapeutic potential in the treatment of mitochondrial related disorders, such as Parkinson's disease, Alzheimers disease, diabetes, ischemic limb disorder (i.e. as a result of diabetes), hypertension, heart failure, peripheral artery disease, cateracts, oxidative damage due to air pollution, UV and gamma radiation, cancer, and also find use in conjunction with chemotherapy or radiation therapy.

Subjects suitable for treatment with ψεHSP90 peptide include, but are not limited to, individuals who are scheduled to undergo cardiac surgery or who have undergone cardiac surgery; individuals who have experienced a stroke; individuals who have suffered brain trauma; individuals who have prolonged surgery in which blood flow is impaired; individuals who have suffered a myocardial infarct (e.g., acute myocardial infarction); individuals who suffer from cerebrovascular disease; individuals who have spinal cord injury; individuals having a subarachnoid hemorrhage; and individuals who will be subjected to organ transplantation. Subjects suitable for treatment with ψεHSP90 peptide include subjects having an ischemic limb disorder, e.g., resulting from Type 1 or Type 2 diabetes.

In other embodiments, subjects suitable for treatment with ψεHSP90 peptide include, but are not limited to, individuals who are having or who have experienced a seizure; individuals having skin damage resulting from UV exposure; individuals having photodamage of the skin; individuals having an acute thermal skin burn, individuals undergoing radiation therapy (i.e. for cancer treatment) and individuals suffering from tissue hyperoxia.

In still other embodiments, subjects suitable for treatment with ψεHSP90 peptide include, but are not limited to, individuals who have been diagnosed with Alzheimer's disease, Parkinson's disease, amyotrophic lateral sclerosis, or other neurodegenerative disease; individuals having atherosclerosis; individuals having esophageal cancer; individuals having head and neck squamous cell carcinoma; and individuals having upper aerodigestive tract cancer.

Subjects suitable for treatment with a ψεHSP90 peptide additionally include individuals having angina; individuals having heart failure; individuals having hypertension; and individuals having heart disease.

Additional peptide modulators for use in the present composition and method have amino acid sequences similar to the amino acid sequence of ψεHSP90. In some embodiments, the isolated modulator sequences have at least about 50% identity to ψεHSP90. Preferably, the isolated amino acid sequences of the peptide modulators have at least about 60% identity, at least about 70% identity, or at least about 80% identity to the amino acid sequence of ψεHSP90. In particular embodiments, the modulators have at least about 81% identity, at least about 82% identity, at least about 83% identity, at least about 84% identity, at least about 85% identity, at least about 86% identity, at least about 87% identity, at least about 88% identity, at least about 89% identity, at least about 90% identity, at least about 91% identity, at least about 92% identity, at least about 93% identity, at least about 94% identity, at least about 95% identity, at least about 96% identity, at least about 97% identity, at least about 98% identity, and even at least about 99% identity, to isolated ψεHSP90.

Percent identity may be determined, for example, by comparing sequence information using the advanced BLAST computer program, including version 2.2.9, available from the National Institutes of Health. The BLAST program is based on the alignment method of Karlin and Altschul ((1990) Proc. Natl. Acad Sci. USA 87:2264-68) and as discussed in Altschul et al. ((1990) J. Mol. Biol. 215:403-10; Karlin and Altschul (1993) Proc. Natl. Acad. Sci. USA 90:5873-77, and Altschul et al (1997) Nucleic Acids Res. 25:3389-3402).

Conservative amino acid substitutions may be made in the amino acid sequences described herein to obtain derivatives of the peptides that may advantageously be utilized in the present invention. Conservative amino acid substitutions, as known in the art and as referred to herein, involve substituting amino acids in a protein with amino acids having similar side chains in terms of, for example, structure, size and/or chemical properties. For example, the amino acids within each of the following groups may be interchanged with other amino acids in the same group: amino acids having aliphatic side chains, including glycine, alanine, valine, leucine and isoleucine; amino acids having non-aromatic, hydroxyl-containing side chains, such as serine and threonine; amino acids having acidic side chains, such as aspartic acid and glutamic acid, amino acids having amide side chains, including glutamine and asparagine; basic amino acids, including lysine, arginine and histidine; amino acids having aromatic ring side chains, including phenylalanine, tyrosine and tryptophan; and amino acids having sulfur-containing side chains, including cysteine and methionine. Additionally, amino acids having acidic side chains, such as aspartic acid and glutamic acid, are considered interchangeable herein with amino acids having amide side chains, such as asparagine and glutamine. A modulator peptide may also include natural amino acids, such as the L-amino acids or non-natural amino acids, such as D-amino acids,

Particular peptides expected to work in manner similar to ψεHSP90 include PRDNEER (SEQ ID NO. 6), PHDNEER (SEQ ID NO: 7), PKENEER (SEQ ID NO: 8), PKDQEER (SEQ ID NO: 9), PKDNDER (SEQ ID NO: 10), PKDNEDR (SEQ ID NO: 11), PKDNEEK (SEQ ID NO: 12), PKDNEEH (SEQ ID NO: 13), which include single conservative substitutions in the peptide, and PRENEER (SEQ ID NO: 14), PRDQEER (SEQ ID NO: 15), PRDNDER (SEQ ID NO: 16), PRDNEDR (SEQ ID NO: 17), PRDNEEK (SEQ ID NO: 18), PRDNEEH (SEQ ID NO: 19), PHENEER (SEQ ID NO: 20), PHDQEER (SEQ ID NO: 21), PHDNDER (SEQ ID NO: 22), PHDNEDR (SEQ ID NO: 23), PHDNEEK (SEQ ID NO: 24), PHDNEEH (SEQ ID NO: 25), PKDNEER (SEQ ID NO: 26), PKEQEER (SEQ ID NO: 27), PKENDER (SEQ ID NO: 28), PKENEDR (SEQ ID NO: 29), PKENEEK (SEQ ID NO: 30), PKENEEH (SEQ ID NO. 31), PRENEER (SEQ ID NO: 32), PHENEER (SEQ ID NO: 33), PKEQEER (SEQ ID NO: 34), PKENDER (SEQ ID NO: 35), PKENEDR (SEQ ID NO: 36), PKENEEK (SEQ ID NO: 37), PKENEEH (SEQ ID NO: 38), PRDQEER (SEQ ID NO: 39), PHDQEER (SEQ ID NO: 40), PKEQEER (SEQ ID NO: 41), PKDQDER (SEQ ID NO: 42), PKDQEDR (SEQ ID NO: 43), PKDQEEK (SEQ ID NO: 44), PKDQEEH (SEQ ID NO: 45), PRDNDER (SEQ ID NO: 46), PHDNDER (SEQ ID NO: 47), PKENDER (SEQ ID NO: 48), PKDQDER (SEQ ID NO: 49), PKDNDDR (SEQ ID NO: 50), PKDNDEK (SEQ ID NO: 51), PKDNDEH (SEQ ID NO: 52), PRDNEDR (SEQ ID NO: 53), PHDNEDR (SEQ ID NO: 54), PKENEDR (SEQ ID NO: 55), PKDQEDR (SEQ ID NO: 56), PKDNDDR (SEQ ID NO: 57), PKDNEDK (SEQ ID NO: 58), PKDNEDH (SEQ ID NO: 59), PRDNEEK (SEQ ID NO: 60), PHDNEEK (SEQ ID NO: 61), PKENEEK (SEQ ID NO: 62), PKDQEEK (SEQ ID NO: 63), PKDNDEK (SEQ ID NO: 64), PKDNEDK (SEQ ID NO: 65), PRDNEEH (SEQ ID NO: 66), PHDNEEH (SEQ ID NO: 67), PKENEEH (SEQ ID NO: 68), PKDQEEH (SEQ ID NO: 69), PKDNDEH (SEQ ID NO: 70), PKDNEDH (SEQ ID NO: 71), which include two conservative substitutions in each peptide.

The exemplified ψεHSP90 peptide is a heptamer (i.e., 7 amino acid residues in length). However, shorter peptides, e.g., pentamers and hexamers, that include a portion of the described heptamer sequence or related sequences, are expected to produce similar results. Such preferred pentamers and hexamers may include the P and the adjacent K, N, or H, which are present in the exemplified heptamers. Yet further peptides may include, in addition to the sequences indicated, upstream and/or downstream flanking amino acid residues from the V2 region of εPKC (amino acid residues 130-153), which is conserved in murine, rat, and human εPKC (FIG. 4C; SEQ ID NO: 74).

Yet further peptides are derived from other portions of the V2 domain and do not include the above described peptide. Accordingly, peptides for use as described may be from about 5 to about 30, from about 6 to about 20, from about 7 to about 15, or even from about 8 to about 12 amino acid residues in length, and derived from the V2 domain of εPKC. Exemplary lengths are 5, 6, 7, 8, 9, 10, 11, 12, 13, 14, 15, 16, 17, 18, 19, 20, 21, 22, 23, 24, 25, 26, 27, 28, 29, and 30 amino acids. Note that any subset of these peptides may be expressly specified or excluded (as in the case of a proviso) in a genus of peptides.

In addition, a wide variety of modifications to the amide bonds which link amino acids may be made and are known in the art. Such modifications are discussed in general reviews, including in Freidinger, R. M. (2003) J. Med. Chem. 46:5553, and Ripka, A. S. and Rich, D. H. (1998) Curr. Opin, Chem. Biol. 2:441. These modifications are designed to improve the properties of the peptide by increasing the potency of the peptide or by increasing the half-life of the peptide.

The peptide modulators may be pegylated, which is a common modification to reduce systemic clearance with minimal loss of biological activity. Polyethylene glycol polymers (PEG) may be linked to various functional groups of peptide modulators using methods known in the art (see, e.g., Roberts et al. (2002), Advanced Drug Delivery Reviews 54:459-76 and Sakane et al. (1997) Pharm. Res. 14:1085-91). PEG may be linked to, e.g., amino groups, carboxyl groups, modified or natural N-termini, amine groups, and thiol groups. In some embodiments, one or more surface amino acid residues are modified with PEG molecules. PEG molecules may be of various sizes (e.g., ranging from about 2 to 40 kDa). PEG molecules linked to modulator peptides may have a molecular weight about any of 2,000, 10,000, 15,000, 20,000, 25,000, 30,000, 35,000, 40,000 Da. PEG molecule may be a single or branched chain. To link PEG to modulator peptides, a derivative of PEG having a functional group at one or both termini may be used. The functional group is chosen based on the type of available reactive group on the polypeptide. Methods of linking derivatives to polypeptides are known in the art.

In some embodiments, the peptide modulator is modified with to achieve an increase in cellular uptake. Such a modification may be, for example, attachment to a carrier peptide, such as a Drosophila melanogaster Antennapedia homeodomain-derived sequence (unmodified sequence may be found in Genbank Accession No. AAD19795) which is set forth in SEQ ID NO: 72 (RQIKIWFQNRRMKWKK), the attachment being achieved, for example, by cross-linking via an N-terminal Cys-Cys bond as discussed in Theodore, L., et al. J. Neurosci. 15:7158-7167 (1995); Johnson, J. A., et al. Circ. Res 79:1086 (1996). The terminal cysteine residues may be part of the naturally-occurring or modified amino acid sequences or may be added to an amino sequence to facilitate attachment. The carrier peptide sequence may also be sought from Drosophila hydei and Drosophila virilis. Alternatively, the peptide modulator may be modified by a Transactivating Regulatory Protein (Tat)-derived transport polypeptide (such as from amino acids 47-57 of Tat shown in SEQ ID NO: 73; YGRKKRRQRRR) from the Human Immunodeficiency Virus, Type 1, as described in Vives, et al., J. Biol. Chem, 272:16010-16017 (1997), U.S. Pat. No, 5,804,604; and as seen in Genbank Accession No. AAT48070, or with polyarginine as described in Mitchell, et al. J. Peptide Res. 56:318-325 (2000) and Rothbard, et al., Nature Med. 6:1 253-1257 (2000). The peptide modulator may be modified by other methods known to the skilled artisan in order to increase the cellular uptake of therapeutic agents into the mitochondria.

Peptide modulators may be obtained by methods known to the skilled artisan. For example. The peptide modulators may be chemically synthesized using various solid phase synthetic technologies known to the art and as described, for example, in Williams, Paul Lloyd, et al. Chemical Approaches to the Synthesis of Peptides and Proteins, CRC Press, Boca Raton, Fla. (1997).

Alternatively, the modulators may be produced by recombinant technology methods as known in the art and as described, for example, in Sambrook et al., Molecular Cloning: A Laboratory Manual, Cold Springs Harbor laboratory, 2^{nd} ed., Cold Springs Harbor, N.Y. (1989), Martin, Robin, Protein Synthesis: Methods and Protocols, Humana Press, Totowa, N.J. (1998) and Current Protocols in Molecular Biology (Ausubel et al., eds.), John Wiley & Sons, which is regularly and periodically updated. An expression vector may be used to produce the desired peptide modulator in an appropriate host cell and the product may then be isolated by known methods. The expression vector may include, for example, the nucleotide sequence encoding the desired peptide wherein the nucleotide sequence is operably linked to a promoter sequence.

While the present treatment method has largely been described in terms of peptide modulators, the method includes administering to an animal in need of such treatment a polynucleotide encoding any of the modulators described herein. Polynucleotide encoding peptide modulators include gene therapy vectors based on, e.g., adenovirus, adeno-associated virus, retroviruses (including lentiviruses), pox virus, herpesvirus, single-stranded RNA viruses (e.g., alphavirus, flavivirus, and poliovirus), etc. Polynucleotide encoding peptide modulators further include naked DNA or plasmids operably linked to a suitable promoter sequence and suitable of directing the expression of the peptides. Polypeptides may be encoded by an expression vector, which may include, for example, the nucleotide sequence encoding the desired peptide wherein the nucleotide sequence is operably linked to a promoter sequence.

As defined herein, a nucleotide sequence is “operably linked” to another nucleotide sequence when it is placed in a functional relationship with another nucleotide sequence. For example, if a coding sequence is operably linked to a promoter sequence, this generally means that the promoter may promote transcription of the coding sequence. Operably linked means that the DNA sequences being linked are typically contiguous and, where necessary to join two protein coding regions, contiguous and in reading frame. However, since enhancers may function when separated from the promoter by several kilobases and intronic sequences may be of variable length, some nucleotide sequences may be operably linked but not contiguous. Additionally, as defined herein, a nucleotide sequence is intended to refer to a natural or synthetic linear and sequential array of nucleotides and/or nucleosides, and derivatives thereof. The terms “encoding” and “coding” refer to the process by which a nucleotide sequence, through the mechanisms of transcription and translation, provides the information to a cell from which a series of amino acids can be assembled into a specific amino acid sequence to produce a polypeptide.

Other suitable modulators include organic or inorganic compounds, such as peptidomimetic small-molecules.

### IV. Administration and Dosing of PKC Modulators

Modulators of εPKC-HSP90 interactions may be administered to a patient by a variety of routes. For example, the modulators may be administered parenterally, including intraperitoneally; intravenously; intraarterially; subcutaneously, or intramuscularly. The modulators may also be administered via a mucosal surface, including rectally, and intravaginally; intranasally; by inhalation, either orally or intranasally; orally, including sublingually; intraocularly and transdermally. Combinations of these routes of administration are also envisioned,

The modulators may also be administered in various conventional forms. For example, the modulators may be administered in tablet form for sublingual administration, in a solution or emulsion. The modulators may also be mixed with a pharmaceutically-acceptable carrier or vehicle In this manner, the modulators are used in the manufacture of a medicament treating various diseases and disorders.

The vehicle may be a liquid, suitable, for example, for parenteral administration, including water, saline or other aqueous solution, or may be an oil or an aerosol. The vehicle may be selected for intravenous or intraarterial administration, and may include a sterile aqueous or non-aqueous solution that may include preservatives, bacteriostats, buffers and antioxidants known to the art. In the aerosol form, the modulator may be used as a powder, with properties including particle size, morphology and surface energy known to the art for optimal dispersability. In tablet form, a solid vehicle may include, for example, lactose, starch, carboxymethyl cellulose, dextrin, calcium phosphate, calcium carbonate, synthetic or natural calcium allocate, magnesium oxide, dry aluminum hydroxide, magnesium stearate, sodium bicarbonate, dry yeast or a combination thereof. The tablet preferably includes one or more agents which aid in oral dissolution. The modulators may also be administered in forms in which other similar drugs known in the art are administered, including patches, a bolus, time release formulations, and the like.

The modulators described herein may be administered for prolonged periods of time without causing desensitization of the patient to the therapeutic agent. That is, the modulators can be administered multiple times, or after a prolonged period of time including one, two or three or more days; one two, or three or more weeks or several months to a patient and will continue to cause an increase in the flow of blood in the respective blood vessel

Suitable carriers, diluents and excipients are well known in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound of the present invention is being applied. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. Formulations may also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e.,, a compound of the present invention or pharmaceutical composition thereof) or aid in the manufacturing of the pharmaceutical product (i.e., medicament). Some formulations may include carriers such as liposomes. Liposomal preparations include, but are not limited to, cytofectins, multilamellar vesicles and unilamellar vesicles. Excipients and formulations for parenteral and nonparenteral drug delivery are set forth in Remington, The Science and Practice of Pharmacy (2000).

The skilled artisan will be able to determine the optimum dosage. Generally, the amount of modulator utilized may be, for example, about 0.0005 mg/kg body weight to about 50 mg/kg body weight, but is preferably about 0.05 mg/kg to about 0.5 mg/kg. The exemplary concentration of the modulator used herein are from 3 mM to 30 mM but concentrations from below about 0.01 mM to above about 100 mM (or to saturation) are expected to provide acceptable results.

The modulators may also be delivered using an osmotic pump. An osmotic pump allows a continuous and consistent dosage of modulator to be delivered to an animal with minimal handling.

### V. Compositions and Kits Comprising Modulators of εPKC-HSP90 Interactions

The methods may be practiced using peptide and/or peptidomimetic modulators of εPKC-HSP90interactions, some of which are identified herein. These compositions may be provided as a formulation in combination with a suitable pharmaceutical carrier, which encompasses liquid formulations, tablets, capsules, films, etc. The modulators may also be supplied in lyophilized form. The compositions are suitable sterilized and sealed for protection.

Such compositions may be a component of a kit of parts (tie., kit). Such kits may further include administration and dosing instructions, instructions for identifying patients in need of treatment, and instructions for monitoring a patients' response to therapy. Where the modulator is administered via a pump, the kit may comprise a pump suitable for delivering the modulator. The kit may also contain a syringe to administer a formulation comprising a modulator by a peripheral route.

The foregoing description and the following examples are not intended to be limiting. Further aspects and embodiments of the compositions and methods will be apparent to the skilled artisan in view of the present teachings.

### Examples

The following examples are illustrative in nature and are in no way intended to be limiting.

### Materials

All antibodies used were obtained from Santa Cruz Biotechnology, with the exception of the VDAC antibody (MitoSciences) and the Na/K_{ATP}ase antibody (Upstate Biotechnology). Protein A/G beads used for immunoprecipitation were from Santa Cruz Biotechnology. Secondary horseradish peroxidase-conjugated antibodies were from Amersham Biosciences. The gold-conjugated secondary antibody used for immunogold electron microscopy was from Ted Pella, Inc. The ψεHSP90 peptide (PKDNEER) was synthesized and conjugated to TAT₄₇₋₅₇ by American Peptide Company, Inc (Sunnyvale, Calif.). The HSP90 inhibitor geldanamycin, was purchased from InvivoGen.

### Methods

### Ex Vivo Model of Cardiac Ischemia-Reperfusion Injury Using the Langendorff Perfused Rat Heart Model

Rat hearts (Wistar, 250-300 g) were excised and cannulated on a Langendorff apparatus via the aorta. Briefly, retrograde perfusion was carried out with a constant coronary flow rate of 10 ml/min with oxygenated Krebs-Henseleit buffer containing 120 mM NaCl, 5,8 mM KCl, 25 mM NaHCO3, 1.2 mM NaHCO3, 1.2 mM MgSO4, 1.2 mM CaCl2, and 10 mM dextrose, pH 7.4 at 37° C. After a 20 min equilibration period, hearts were subjected to a 35 min of no-flow, global ischemia followed by 15 min of reperfusion. Control hearts were perfused with normoxic Krebs buffer and were time-matched for the experimental period. The cardiac perfusate was collected throughout the duration of reperfusion and measurement of cardiac damage by creatine phosphokinase (CPK) release was carried out using a standard assay kit (Equal Diagnostics, CT, USA).Where indicated, the HSP90 inhibitor, geldanamycin (5 μM or 10 μM), was perfused for the duration of the reperfusion period and the ψεHSP90 peptide (1 μm), was perfused for 10 min prior to 35 min of ischemia and during the entire 15 min reperfusion period. At the end of the 15 mins reperfusion period, hearts were rapidly transferred to ice-cold homogenization buffer and subcellular fractions separated by differential centrifugation as described below.

### Heart Tissue Fractionation and Western Blot Analysis

Rat heart ventricles were removed from the cannula and homogenized in ice-cold mannitol-sucrose buffer (210 mM mannitol, 70 mM sucrose, 5 mM MOPS and 1 mM EDTA containing Sigma Protease Inhibitor 1 and Sigma Phosphatase Inhibitors 1 and 2, added per manufacturer's instructions) The resultant homogenate was filtered through gauze and centrifuged at 700 g for 5 minutes to pellet nuclei and cellular debris. The supernatant was centrifuged at 10,000 g to pellet the mitochondrial-enriched fraction and the supernatant from this fraction centrifuged at 100,000 g to pellet the plasma membrane fraction. The final supernatant was the cytosolic fraction Fractional purity was demonstrated using protein markers (GAPDH for cytosol, β-integrin or Na/KATPase for plasma membrane, ALDH2, VDAC or ANT for mitochondria). Mitochondrial purity was also assessed by electron microscopic analysis. Protein concentration was determined by Bradford assay and 10 μg protein separated on a 12% SDS-PAGE gel and transferred to nitrocellulose. PKC translocation was measured using antibodies raised against εPKC and δPKC with mitochondrial (ANT) and cytosolic (GAPDH) markers used to ensure equal sample loading of the mitochondrial and cytosolic fractions, respectively. Densitometry was performed using Image J software (NIH).

### Immunoprecipitation

200-500 μg of mitochondrial or cytosolic protein was diluted into 1 ml of IP Lysis Buffer (150 mM NaCl, 10 mM Tris-HCl, 5 mM EDTA, 0.1% Triton X-100, pH=7.4, containing Sigma Protease Inhibitor 1 and Sigma Phosphatase Inhibitors 1 and 2). Proteins were incubated with εPKC antibody (2 μg) for 2 hours at 4° C. with inversion mixing. 10 μg of Protein A/G Beads (Santa Cruz Biotechnology) were added and the mixture was incubated overnight at 4° C. with inversion mixing. Beads were then centrifuged, washed 3× in IP Lysis Buffer, then re-suspended in sample buffer. Immunoprecipitated proteins were separated on 10% SDS-PAGE gels. The presence of associated proteins was determined by western blotting using antibodies specific for PKCε HSP90, TOM20 and TIM23. Protein immunoprecipitation was then repeated in reverse (immunoprecipitation with HSP90, TOM20 or TIM23 antibodies followed by western blotting with εPKC antibody). A beads alone group was also included for each sample set to assess any non-specific protein binding to the beads.

### Mitochondrial Sub-Fractionation

Submitochondrial particles (SMP) were generated. Briefly, isolated cardiac mitochondria were resuspended to a final volume of 10 mg/ml in ice-cold mannitol-sucrose buffer (210 mM mannitol, 70 mM sucrose, 5 mM MOPS and 1 mM EDTA) then sonicated 3×2 min on ice with 1 min intervals. The solution was then spun at 10,000 g for 10 min to pellet unbroken mitochondria. The supernatant was then spun at 100,000 g for 30 min to pellet SMPs. For trypsin digestion of proteins, 100 μg SMPs were incubated with 1 μg trypsin protease (Sigma) in 100 μl MS buffer for 0, 5, 10 and 20 minutes at 37° C. The trypsin digestion was quenched by adding protease inhibitor cocktail (1 μl) and SMPs were pelleted by centrifucation at 10,000 g for 10 min. For high salt treatment, 100 μl of 1 mg/ml SMPs were incubated with 100 μl of 400 mM KCl with mild shaking on ice for 10 minutes followed by centrifugation. For high pH treatment, 100 μl of 1 mg/ml SMPs were incubated with 200 mM Na₂CO₃ (pH 11.5) for 10 minutes followed by centrifugation. Recovered SMP pellets were then resuspended in 50 μl sample loading buffer and proteins separated by SDS-PAGE.

In order to isolate inner mitochondrial components, freshly isolated cardiac mitochondria (50 μl of 10 μg/μl) were re-suspended in 450 μl hypotonic buffer (5 mM Tris-HCl, 1 mM EDTA pH 7.4) and incubated on ice for 15 minutes. Exposure to hypotonic buffer results in matrix swelling and rupture of the outer mitochondrial membrane. The resultant solution was centrifuged at 20,000 g for 10 min at 4° C. to pellet the mitoplasts (consisting of the inner mitochondrial membrane (IMM) and matrix) while the supernatant contains the outer membrane (OM) and the intermembrane space (IMS). Mitoplasts were then incubated in 450 μl of potassium phosphate buffer (1 mM potassium phosphate pH 7.4) and sonicated 3×2 min on ice with 1 min intervals to disrupt the mitochondrial inner membrane (IMM). The solution was then spun at 100,000 g for 40 min. The resultant pellet contains the IMM and the matrix proteins remain in the supernatant. Western blotting for the presence of εPKC was performed in parallel with western blotting with antibodies specific for proteins that localize to distinct mitochondrial sub-compartments including the IMM [adenine nucleoside transporter (ANT)] the matrix [aldehyde dehydrogenase 2 (ALDH2) or glucose related protein (GRP-75)] and the IMS [cytochrome C (Cyt-c)].

### In Vitro Mitochondrial Translocation Assay

Activation of recombinant εPKC was performed as described in the art. Briefly, recombinant εPKC (5 μl of 10 ng/μl stock) (Cell Signaling Technology) was aliquoted into assay buffer (20 mM Tris HCl, 50 mM KCl, 1 mM DTT, 0.1 mg/ml BSA Mg²⁺ (5 mM), ATP (100 μM), pH 7.4) containing different combinations of activation factors including phosphatidylserine/diacylglycerol (1 mM), H₂O₂ (50 μM) or ψεHSP90 (1 μm) in a final volume of 50 μl . εPKC was activated by incubation in assay buffer at 37° C. for 20 min. Rabbit reticulocyte lysate (RRL) (10 μl) was added to the incubation mixture for 10 minutes after εPKC activation. The activated recombinant εPKC mixture was then added to freshly isolated cardiac mitochondria (1 mg/ml) in 500 μl mitochondrial translocation assay (MTA) buffer (250 mM sucrose, 80 mM KCl, 5 mM MgCl₂, 2 mM KH₂PO₄, 10 mM MOPS-KOH, 10 mM succinate, 2 mM ATP, 3% BSA, pH 7.2) and incubated for 20 minutes at 37° C. with shaking (for ψεHSP90 treated groups, 1 μM ψεHSP90 was also present in the MTA buffer). The reaction was halted by the addition of 50 μM dinitrophenol (DNP) to destroy the mitochondrial membrane potential which is required for mitochondrial import. Mitochondria were then spun at 10,000 g×10 min, resuspended in 50 μl sample loading buffer and mitochondrial εPKC levels measured by western blotting using VDAC as a loading control. Recombinant GST-εPKC has a molecular weight of ˜115 kD and was thus distinguished from any endogenous εPKC (molecular weight ˜90 kD) that is intrinsic to the mitochondria.

### Immunogold Electron Microscopy

Freshly isolated cardiac mitochondria were fixed overnight in 4% paraformaldehyde and 0.025% gluteraldehyde. The fixed material was sectioned by the Stanford Electron Microscopy Facility. Ultrathin sections of between 75 and 80 nm were mounted on formvar/carbon coated 75 mesh Ni grids. Grids were incubated for 1 hour at room temperature in blocking solution [140 mM NaCl, 3 mM KCl, 8 mM Na2HPO4, 1.5 mMKH2PO4, 0.05% Tween-20, pH7.4 containing 0.5%(w/v) ovalbumin (Sigma), 0.5%(w/v) BSA (Sigma)]. Grids were then incubated for 1 hour with anti-εPKC antibody (rabbit polyclonal) (Santa Cruz, Calif.) (1:100 in blocking solution) followed by 3×15 min washes in PBST (140 mM NaCl, 3 mM KCl, 8 mM Na2HPO4, 1.5 mMKH2PO4, 0.05% Tween-20, pH7,4) followed by 1 hour incubation with goat anti-rabbit IgG conjugated to 10 nm gold particles (Ted Pella Inc) (1:100 in blocking solution). Grids were then washed 3×15 min in PBST and stained for 20 s in 1:1 saturated uranylacetate (7.7%) in acetone followed by staining in 0.2% lead citrate for 3 to 4 min for contrast. Mitochondria were observed in a JEOL 1230 transmission electron microscope at 80 kV and photos were taken using a Gatan Multiscan 791 digital camera.

### Sequence Alignments

Sequences of the human PKC family members (GenBank™ accession numbers: αPKC; NP_{—}002728.1, βPKC; NP_{—}002729.2, γPKC; EAW72161,1, δPKC; NP_{—}997704.1, εPKC; NP_{—}005391.1, θPKC; NP_{—}006248.1, ηPKC; NP_{—}006246.2 and ζPKC; CAA78813.1) and of εPKC of various species (human εPKC; NP_{—}005391.1, rat; NP_{—}058867.1 mouse; NP_{—}035234.1 and Xenopus; NP_{—}001107724.1) were aligned using ClustalW software. The sequences of human εPKC (accession number NP_{—}005391.1) was aligned with human HSP90α (accession number NP_{—}005339) and HSP90β (accession number; NP_{—}031381) using LALIGN software.

### Statistics

Data are expressed as Mean±Standard Error of the Mean (SEM). Statistical significance was calculated between groups using the student's t test (p<0.05 was considered statistically significant).

### Example 1

### HSP90 Inhibition Prevents Mitochondrial Translocation of εPKC and Increases Cardiac Injury Induced by Ischemia Reperfusion

Langendorf-perfused rat hearts were subjected to 35 minutes of ischemia and 15 minutes of reperfusion (I₃₅/R₁₅) in the presence and absence of the HSP90 inhibitor, geldanamycin (GA; 5 μm) for the first 10 minutes of reperfusion (FIG. 1A). Necrotic damage was determined by measurement of the release of the cardiac myocyte cytosolic enzyme, creatine phosphokinase (CPK), into the effluent (FIG. 1B). The levels are expressed in arbitrary CPK units. I₃₅/R₁₅ resulted in an about 8.5-fold increase in necrotic injury compared to hearts that were not subjected to I₁₃/R₁₅ (n=7; * denotes p<0.05). Inhibition of HSP90 with GA further increased injury, as evidenced by a 176% increase in CPK release (n=7; p<0.05). Treatment of hearts that were not subjected to I₁₃₅/R₁₅ with increasing concentrations of GA did not result in any significant release in CPK release into the cardiac effluent (FIG. 1B).

Following Langendorff perfusion, hearts were removed, homogenized, fractionated into mitochondrial and cytosolic components, Western blotted (WB) with antibodies specific to ε and δPKC and normalized to ANT (mitochondrial fraction) and GAPDH (cytosolic fraction). εPKC and δPKC translocation to the mitochondria was quantified and results are displayed in a histogram and expressed as arbitrary units. Hearts that were subjected to I₁₃₅/R₁₅ had a 169% increase in δPKC, as seen in FIG. 1E, and a 438% increase in εPKC in the mitochondrial fraction, as seen in FIG. 1D (p<0.01; n=5). Inhibition of HSP90 with GA blocked the translocation of εPKC by 54% but did not affect δPKC translocation to the mitochondrial fraction (FIGS. 1D-1E, p<0.05; n=5).

### Example 2

### Interaction of εPKC with Mitochondrial Import Proteins

Hearts were perfused in Langendorf mode as described above, after which they were homogenized, fractionated and subjected to immunoprecipitation analysis with the antibodies listed in the figures. As shown in FIG. 2A, there was no association between εPKC and HSP90 in the cytosolic fraction in any of the conditions tested. However, in the mitochondrial fraction there was a significant association between εPKC and HSP90 that was inhibited by treatment with the HSP90 inhibitor GA (5 μM), seen in the upper panel of FIG. 2B (n=3). No association between HSP90 and εPKC was observed under basal (i.e., non-ischemic) conditions in the mitochondrial fraction. However, when hearts were subjected to IR, εPKC co-immunoprecipitated with HSP90 in the mitochondrial fraction and this association was blocked by treatment with 5 μm GA (FIG. 2B, upper panel; n=3). This association was confirmed by reverse immunoprecipitation (FIG. 2B, lower panel; n=3). As shown, substantial co-immunoprecipitation of Tom20 and εPKC was observed following exposure to IR, which was abolished by 5 μm GA (FIG. 2C; n=3). A similar HSP90-dependent interaction of εPKC with Tim23 was observed following IR; i.e., association of PKC with Tim 23 substantially decreased following IR when hearts were treated with GA (FIG. 2D,; n=3). These results suggest that εPKC interacts with components of the mitochondrial import machinery in an HSP90-dependent manner following IR.

### Example 3

### Mitochondrial Translocation of εPKC Following Ischemia and Reperfusion

Hearts were perfused in Langendorf mode as described above, after which they were homogenized, fractionated and the mitochondria were fixed, sectioned onto nickel grids, incubated with εPKC-specific antibody and gold conjugated secondary antibody and visualized by electron microscopy (FIG. 3A). Each black dot represents an antibody labeled with gold particle that is bound to εPKC; the average number of dots per mitochondria were counted by an observer blinded to the experimental conditions and displayed as a histogram. To determine non-specific binding, grids were incubated with the secondary gold-conjugated antibody alone (lower right panel, “2° Alone”). There was some εPKC present in hearts subjected to normoxia (no IR) (upper left panel, “Normoxia”), but the amount increased by 2.5 fold following IR (upper right panel, “IR”, quantified in histogram of FIG. 3B). Treatment with 5 μm GA completely blocked IR-induced accumulation of εPKC within cardiac mitochondria (lower left panel “IR+GA”, quantified in histogram of FIG. 3B).

For further localization analysis of εPKC within the mitochondria, mitochondria were subfractionated into inner mitochondrial membrane (IMM), matrix, and inter mitochondrial membrane space (IMS) components by hypotonic treatment and centrifugation (FIG. 3C). Fraction purity was assessed with antibodies against ANT (a marker of IMM), Grp75 (a marker of matrix fraction), and enolase (a cytosolic enzyme; FIG. 3D). εPKC localization in the IMM fraction following IR increased by ˜10 fold and, similar to the electron microscopic analysis, was completely blocked by GA treatment (n=3). Quantification of εPKC localization at the IMM (n=3; p<0.05) is shown in FIG. 3E. To determine how εPKC associates with the IMM, inside-out sub-mitochondrial particles were generated from isolated mitochondria, which exposes proteins that associated with IMM which face the matrix while sequester proteins that face the IMS within the inverted mitochondrial vesicle. These vesicles were then treated with base (pH 11.5), high salt (400 mM KCl), and with trypsin. Exposure to carbonate wash at pH 11.5 (used to remove strongly associated, membrane-associated proteins) removed εPKC from the IMM, whereas exposure to 400 mM KCl high-salt wash (used to remove loosely associated proteins) did not (FIG. 3F, upper left panel). These findings suggest a tight interaction between εPKC and the IMM. Treatment with the protease trypsin (which cannot cross the membrane) degraded εPKC (FIG. 3F, upper right panel), but did not affect levels of cytochrome c (FIG. 3F lower right panel). That trypsin could access εPKC suggests that εPKC is present on the matrix side of the IMM, which is exposed to trypsin in the SMP preparation.

### Example 4

### Administration of ψεHSP90 Peptide to Hearts Exposed to Ischemia and Reperfusion

Isolated rat hearts were perfused with oxygenated Krebs-Henseleit solution comprised of NaCl (120 nmol/L); KCl (5.8 nmol/L); NaHCO₃ (25 nmol/L); NaH₂O₄ (1.2 nmol/L); MgSO₄ (1.2 nmol/L); CaCl₂ (1.0 nmol/L); and dextrose (10 nmol/L), pH 7.4 at 37 C. After a 10 minute equilibration period, the hearts were treated with the ψεHSP90 peptide or control buffer solution. As depicted in FIG. 5A, Perfusion was maintained at a constant flow of 10 mL/min with Krebs-Henseleit solution containing 1 μM of the appropriate peptide. The Langendorff method employs retrograde flow from the ventricle to the aorta and into the coronary arteries, bypassing the pulmonary arteries. To induce global ischemia, flow was interrupted for 35 minutes. After the ischemic event, the hearts were reperfused with Krebs-Henseleit solution for 15 minutes.

During reperfusion, ischemia-induced cell damage was determined by measuring the activity of creatine phosphokinase (CPK) in the coronary perfusate, carried out using a standard assay kit (Equal Diagnostics, CT, USA). The ψεHSP90 peptide was perfused in Langendorf mode for 10 minutes before and 10 minutes after the ischemic period (1 micromolar) and myocardial injury was assayed by the release of CPK into the effluent during reperfusion (FIG. 5B). Similar to the previous results, hearts that were subjected to IR had high levels of CPK in the effluent; however, perfusion with ψεHSP90 peptide decreased CPK release by 47% (n4; p<0.05; FIG. 5B). Translocation of εPKC and the related isoform δPKC to the mitochondria and plasma membrane was determined by western blotting using εPKC and δPKC antibodies (Santa Cruz Biotechnology) (FIG. 5C). Treatment with ψεHSP90 enhanced mitochondrial translocation of PKCε to the mitochondrial fraction but not the plasma membrane. Treatment with ψεHSP90 was without effect on δPKC. Physical interaction between PKCε and HSP90 was determined by co-immunoprecipitation of PKCε and HSP90 in the mitochondrial fraction in hearts exposed to normoxia or IR in the absence or presence of 1 μM ψεHSP90 (FIG. 5D). Treatment with ψεHSP90 increased IR-induced physical association of PKCε and HSP90 at the mitochondria and this was reduced by co-administration of the HSP90 inhibitor geldanamycin. These data demonstrate the ψεHSP90 peptide enhances mitochondrial translocation of PKCε and enhances interaction between PKCε and the chaperone protein HSP90, at the mitochondria, in response to ischemia-reperfusion.

### Example 5

### In vitro Testing of ψεHSP90 Peptide to Determine Activation Conditions for εPKC Mitochondrial Import

Cardiac mitochondria isolated from anesthetized non-treated animals were incubated in vitro with the differentially activated εPKC and mitochondrial proteins were probed with anti-εPKC antibody and anti-VDAC antibody. Activation of εPKC with the phospholipids phosphatidylserine (PS) and diacylglycerol (DAG) were required to induce mitochondrial translocation of εPKC (FIG. 6A, lanes 2, 3, 4) and the absence of PS/DAG precludes mitochondrial translocation of εPKC (lane 1, 5, 6). The ψεHSP90 peptide increased mitochondrial εPKC translocation (FIG. 6B, lanes 2, 3, 4, 5) and ψεHSP90 treatment was sufficient to induce mitochondrial association of εPKC in the absence of PS/DAG (lane 5).

Although the peptides and methods have been described with respect to their particular embodiments, it will be apparent to those skilled in the art that various changes and modifications can be made without departing from the invention.

What is claimed is:

1. A peptide consisting of an amino acid sequence that is at least 80% identical to the amino acid sequence of P-K-D-N-E-E-R (SEQ ID NO: 1).

2. The peptide of claim 1, wherein the peptide is at least 90% identical to the amino acid sequence of P-K-D-N-E-E-R (SEQ ID NO: 1).

3. The peptide of claim 1, wherein the peptide is at least 95% identical to the amino acid sequence of P-K-D-N-E-E-R (SEQ ID NO. 1).

4. The peptide of claim 1 attached to a carrier to facilitate transport through a cell membrane or into a mitochondria.

5. A pharmaceutical composition comprising a peptide of claim 1 and a suitable pharmaceutical excipient.

6. A peptide consisting of a sequence of amino acids having at least 80% sequence identity to a contiguous sequence of between 5-15 amino acids residues of the V2 region of epsilon-PKC (SEQ ID NO: 95).

7. The peptide of claim 6 attached to a carrier to facilitate transport through a cell membrane or into a mitochondria.

8. A method for treating a mitochondria-related disorder in a subject, comprising: administering to the subject an isolated peptide consisting of a sequence of amino acid residues having 80% sequence identity to a contiguous sequence of 6-20 amino acid residues from the V2 region of epsilon PKC (SEQ ID NO: 95), wherein the administering of the peptide modulates translocation of epsilon-PKC to the mitochondria, thereby reducing symptoms of the mitochondria-related disorder.

9. A method for reducing cell damage following ischemic reperfusion, comprising: administering to the subject an isolated peptide consisting of a sequence of amino acid residues having 80% sequence identity to a contiguous sequence of 6-20 amino acid residues from the V2 region of epsilon PKC (SEQ ID NO: 95), wherein the administering of the peptide modulates translocation of epsilon-PKC to the mitochondria, thereby reducing cell damage.

10. A method for reducing cell damage mediated by oxidative stress, comprising: administering to the subject an isolated peptide consisting of a sequence of amino acid residues having 80% sequence identity to a contiguous sequence of 6-20 amino acid residues from the V2 region of epsilon PKC (SEQ ID NO: 95), wherein the administering of the peptide modulates translocation of epsilon-PKC to the mitochondria, thereby reducing cell damage.

11. A method for treating a mitochondria-related disorder in a subject, comprising: administering to the subject an isolated peptide consisting of a sequence of amino acid residues having 80% sequence identity to a contiguous sequence of 6-20 amino acid residues from the V2 region of epsilon PKC (SEQ ID NO: 95), said peptide effective to modulate the HSP90-dependent translocation of epsilon-PKC to the mitochondria, thereby reducing symptoms of the mitochondria-related disorder.

12. A method for modulating interactions between epsilon-PKC and HSP90 in mitochondria comprising, incubating the mitochondria in the presence of an isolated peptide consisting of a sequence of amino acid residues having 80% sequence identity to a contiguous sequence of 6-20 amino acid residues from the V2 region of epsilon PKC (SEQ ID NO: 95), wherein the incubating modulates intermolecular interactions between epsilon-PKC and HSP90.

13. A method for modulating mitochondrial import, comprising, incubating the mitochondria in the presence of an isolated peptide consisting of a sequence of amino acid residues having 80% sequence identity to a contiguous sequence of 5-20 amino acid residues from the V2 region of epsilon PKC (SEQ ID NO: 95), wherein the incubating modulates mitochondrial import of a cytosolic polypeptide.

14. The method of claim 13, wherein the peptide is a sequence of amino acids having at least 80% sequence identity to a contiguous sequence of between 5-15 amino acids residues of the V2 region of epsilon-PKC (SEQ ID NO: 95)

15. The method of claim 13, wherein the peptide has the amino acid sequence of SEQ ID NO: 1.

16. The method of claim 13, wherein the peptide is attached to a carrier to facilitate transport through a cell membrane or into a mitochondria.


SEQUENCE LISTING (95 sequences)

SEQ ID NO: 1 - protein, 7 aa, Artificial Sequence
  Synthetic peptide
PKDNEER

SEQ ID NO: 2 - protein, 7 aa, Artificial sequence
  Synthetic peptide
PEDEEEK

SEQ ID NO: 3 - protein, 737 aa, Mus musculus
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGKVYVIIDLSGSSGEAPKDNEERVFRERMRPRKRQGAVRRRVHQVNGHKFMATYLRQPTYCSHCRDFIWGVIGKQGYQCQVCTCVVHKRCHELIITKCAGLKKQETPDEVGSQRFSVNMPHKFGIHNYKVPTFCDHCGSLLWGLLRQGLQCKVCKMNVHRRCETNVAPNCGVDARGIAKVLADLGVTPDKITNSGQRRKKLAAGAESPQPASGNSPSEDDRSKSAPTSPCDQELKELENNIRKALSFDNRGEEHRASSATDGQLASPGENGEVRPGQAKRLGLDEFNFIKVLGKGSFGKVMLAELKGKDEVYAVKVLKKDVILQDDDVDCTMTEKRILALARKHPYLTQLYCCFQTKDRLFFVMEYVNGGDLMFQIQRSRKFDEPRSRFYAAEVTSALMFLHQHGVIYRDLKLDNILLDAEGHCKLADFGMCKEGIMNGVTTTTFCGTPDYIAPEILQELEYGPSVDWWALGVLMYEMMAGQPPFEADNEDDLFESILHDDVLYPVWLSKEAVSILKAFMTKNPHKRLGCVAAQNGEDAIKQHPFFKEIDWVLLEQKKIKPPFKPRIKTKRDVNNFDQDFTREEPILTLVDEAIIKQINQEEFKGFSYFGEDLMP

SEQ ID NO: 4 - protein, 737 aa, Rattus norvegicus
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGKVYVIIDLSGSSGEAPKDNEERVFRERMRPRKRQGAVRRRVHQVNGHKFMATYLRQPTYCSHCRDFIWGVIGKQGYQCQVCTCVVHKRCHELIITKCAGLKKQETPDEVGSQRFSVNMPHKFGIHNYKVPTFCDHCGSLLWGLLRQGLQCKVCKMNVHRRCETNVAPNCGVDARGIAKVLADLGVTPDKITNSGQRRKKLAAGAESPQPASGNSPSEDDRSKSAPTSPCDQELKELENNIRKALSFDNRGEEHRASSSTDGQLASPGENGEVRQGQAKRLGLDEFNFIKVLGKGSFGKVMLAELKGKDEVYAVKVLKKDVILQDDDVDCTMTEKRILALARKHPYLTQLYCCFQTKDRLFFVMEYVNGGDLMFQIQRSRKFDEPRSGFYAAEVTSALMFLHQHGVIYRDLKLDNILLDAEGHSKLADFGMCKEGILNGVTTTTFCGTPDYIAPEILQELEYGPSVDWWALGVLMYEMMAGQPPFEADNEDDLFESILHDDVLYPVWLSKEAVSILKAFMTKNPHKRLGCVAAQNGEDAIKQHPFFKEIDWVLLEQKKMKPPFKPRIKTKRDVNNFDQDFTREEPILTLVDEAIVKQINQEEFKGFSYFGEDLMP

SEQ ID NO: 5 - protein, 737 aa, Homo sapiens
MVVFNGLLKIKICEAVSLKPTAWSLRHAVGPRPQTFLLDPYIALNVDDSRIGQTATKQKTNSPAWHDEFVTDVCNGRKIELAVFHDAPIGYDDFVANCTIQFEELLQNGSRHFEDWIDLEPEGRVYVIIDLSGSSGEAPKDNEERVFRERMRPRKRQGAVRRRVHQVNGHKFMATYLRQPTYCSHCRDFIWGVIGKQGYQCQVCTCVVHKRCHELIITKCAGLKKQETPDQVGSQRFSVNMPHKFGIHNYKVPTFCDHCGSLLWGLLRQGLQCKVCKMNVHRRCETNVAPNCGVDARGIAKVLADLGVTPDKITNSGQRRKKLIAGAESPQPASGSSPSEEDRSKSAPTSPCDQEIKELENNIRKALSFDNRGEEHRAASSPDGQLMSPGENGEVRQGQAKRLGLDEFNFIKVLGKGSFGKVMLAELKGKDEVYAVKVLKKDVILQDDDVDCTMTEKRILALARKHPYLTQLYCCFQTKDRLFFVMEYVNGGDLMFQIQRSRKFDEPRSRFYAAEVTSALMFLHQHGVIYRDLKLDNILLDAEGHCKLADFGMCKEGILNGVTTTTFCGTPDYIAPEILQELEYGPSVDWWALGVLMYEMMAGQPPFEADNEDDLFESILHDDVLYPVWLSKEAVSILKAFMTKNPHKRLGCVASQNGEDAIKQHPFFKEIDWVLLEQKKIKPPFKPRIKTKRDVNNFDQDFTREEPVLTLVDEAIVKQINQEEFKGFSYFGEDLMP

SEQ ID NO: 6 - protein, 7 aa, Artificial sequence
  Synthetic peptide
PRDNEER

SEQ ID NO: 7 - protein, 7 aa, Artificial sequence
  Synthetic peptide
PHDNEER

SEQ ID NO: 8 - protein, 7 aa, Artificial sequence
  Synthetic peptide
PKENEER

SEQ ID NO: 9 - protein, 7 aa, Artificial sequence
  Synthetic peptide
PKDQEER

SEQ ID NO: 10 - protein, 7 aa, Artificial sequence
  Synthetic peptide
PKDNDER

SEQ ID NO: 11 - protein, 7 aa, Artificial sequence
  Synthetic peptide
PKDNEDR

SEQ ID NO: 12 - protein, 7 aa, Artificial sequence
  Synthetic peptide
PKDNEEK

SEQ ID NO: 13 - protein, 7 aa, Artificial sequence
  Synthetic peptide
PKDNEEH

SEQ ID NO: 14 - protein, 7 aa, Artificial sequence
  Synthetic peptide
PRENEER

SEQ ID NO: 15 - protein, 7 aa, Artificial sequence
  Synthetic peptide
PRDQEER

SEQ ID NO: 16 - protein, 7 aa, Artificial sequence
  Synthetic peptide
PRDNDER

SEQ ID NO: 17 - protein, 7 aa, Artificial sequence
  Synthetic peptide
PRDNEDR

SEQ ID NO: 18 - protein, 7 aa, Artificial sequence
  Synthetic peptide
PRDNEEK

SEQ ID NO: 19 - protein, 7 aa, Artificial sequence
  Synthetic peptide
PRDNEEH

SEQ ID NO: 20 - protein, 7 aa, Artificial sequence
  Synthetic peptide
PHENEER

SEQ ID NO: 21 - protein, 7 aa, Artificial sequence
  Synthetic peptide
PHDQEER

SEQ ID NO: 22 - protein, 7 aa, Artificial sequence
  Synthetic peptide
PHDNDER

SEQ ID NO: 23 - protein, 7 aa, Artificial sequence
  Synthetic peptide
PHDNEDR

SEQ ID NO: 24 - protein, 7 aa, Artificial sequence
  Synthetic peptide
PHDNEEK

SEQ ID NO: 25 - protein, 7 aa, Artificial sequence
  Synthetic peptide
PHDNEEH

SEQ ID NO: 26 - protein, 7 aa, Artificial sequence
  Synthetic peptide
PKDNEER

SEQ ID NO: 27 - protein, 7 aa, Artificial sequence
  Synthetic peptide
PKEQEER

SEQ ID NO: 28 - protein, 7 aa, Artificial sequence
  Synthetic peptide
PKENDER

SEQ ID NO: 29 - protein, 7 aa, Artificial sequence
  Synthetic peptide
PKENEDR

SEQ ID NO: 30 - protein, 7 aa, Artificial sequence
  Synthetic peptide
PKENEEK

SEQ ID NO: 31 - protein, 7 aa, Artificial sequence
  Synthetic peptide
PKENEEH

SEQ ID NO: 32 - protein, 7 aa, Artificial sequence
  Synthetic peptide
PRENEER

SEQ ID NO: 33 - protein, 7 aa, Artificial sequence
  Synthetic peptide
PHENEER

SEQ ID NO: 34 - protein, 7 aa, Artificial sequence
  Synthetic peptide
PKEQEER

SEQ ID NO: 35 - protein, 7 aa, Artificial sequence
  Synthetic peptide
PKENDER

SEQ ID NO: 36 - protein, 7 aa, Artificial sequence
  Synthetic peptide
PKENEDR

SEQ ID NO: 37 - protein, 7 aa, Artificial sequence
  Synthetic peptide
PKENEEK

SEQ ID NO: 38 - protein, 7 aa, Artificial sequence
  Synthetic peptide
PKENEEH

SEQ ID NO: 39 - protein, 7 aa, Artificial sequence
  Synthetic peptide
PRDQEER

SEQ ID NO: 40 - protein, 7 aa, Artificial sequence
  Synthetic peptide
PHDQEER

SEQ ID NO: 41 - protein, 7 aa, Artificial sequence
  Synthetic peptide
PKEQEER

SEQ ID NO: 42 - protein, 7 aa, Artificial sequence
  Synthetic peptide
PKDQDER

SEQ ID NO: 43 - protein, 7 aa, Artificial sequence
  Synthetic peptide
PKDQEDR

SEQ ID NO: 44 - protein, 7 aa, Artificial sequence
  Synthetic peptide
PKDQEEK

SEQ ID NO: 45 - protein, 7 aa, Artificial sequence
  Synthetic peptide
PKDQEEH

SEQ ID NO: 46 - protein, 7 aa, Artificial sequence
  Synthetic peptide
PRDNDER

SEQ ID NO: 47 - protein, 7 aa, Artificial sequence
  Synthetic peptide
PHDNDER

SEQ ID NO: 48 - protein, 7 aa, Artificial sequence
  Synthetic peptide
PKENDER

SEQ ID NO: 49 - protein, 7 aa, Artificial sequence
  Synthetic peptide
PKDQDER

SEQ ID NO: 50 - protein, 7 aa, Artificial sequence
  Synthetic peptide
PKDNDDR

SEQ ID NO: 51 - protein, 7 aa, Artificial sequence
  Synthetic peptide
PKDNDEK

SEQ ID NO: 52 - protein, 7 aa, Artificial sequence
  Synthetic peptide
PKDNDEH

SEQ ID NO: 53 - protein, 7 aa, Artificial sequence
  Synthetic peptide
PRDNEDR

SEQ ID NO: 54 - protein, 7 aa, Artificial sequence
  Synthetic peptide
PHDNEDR

SEQ ID NO: 55 - protein, 7 aa, Artificial sequence
  Synthetic peptide
PKENEDR

SEQ ID NO: 56 - protein, 7 aa, Artificial sequence
  Synthetic peptide
PKDQEDR

SEQ ID NO: 57 - protein, 7 aa, Artificial sequence
  Synthetic peptide
PKDNDDR

SEQ ID NO: 58 - protein, 7 aa, Artificial sequence
  Synthetic peptide
PKDNEDK

SEQ ID NO: 59 - protein, 7 aa, Artificial sequence
  Synthetic peptide
PKDNEDH

SEQ ID NO: 60 - protein, 7 aa, Artificial sequence
  Synthetic peptide
PRDNEEK

SEQ ID NO: 61 - protein, 7 aa, Artificial sequence
  Synthetic peptide
PHDNEEK

SEQ ID NO: 62 - protein, 7 aa, Artificial sequence
  Synthetic peptide
PKENEEK

SEQ ID NO: 63 - protein, 7 aa, Artificial sequence
  Synthetic peptide
PKDQEEK

SEQ ID NO: 64 - protein, 7 aa, Artificial sequence
  Synthetic peptide
PKDNDEK

SEQ ID NO: 65 - protein, 7 aa, Artificial sequence
  Synthetic peptide
PKDNEDK

SEQ ID NO: 66 - protein, 7 aa, Artificial sequence
  Synthetic peptide
PRDNEEH

SEQ ID NO: 67 - protein, 7 aa, Artificial sequence
  Synthetic peptide
PHDNEEH

SEQ ID NO: 68 - protein, 7 aa, Artificial sequence
  Synthetic peptide
PKENEEH

SEQ ID NO: 69 - protein, 7 aa, Artificial sequence
  Synthetic peptide
PKDQEEH

SEQ ID NO: 70 - protein, 7 aa, Artificial sequence
  Synthetic peptide
PKDNDEH

SEQ ID NO: 71 - protein, 7 aa, Artificial sequence
  Synthetic peptide
PKDNEDH

SEQ ID NO: 72 - protein, 16 aa, Artificial sequence
  Synthetic peptide
RQIKIWFQNRRMKWKK

SEQ ID NO: 73 - protein, 11 aa, Artificial sequence
  Synthetic peptide
YGRKKRRQRRR

SEQ ID NO: 74 - protein, 24 aa, Artificial sequence
  Synthetic peptide
DLSGSSGEAPKDNEERVFRERMRP

SEQ ID NO: 75 - protein, 30 aa, Artificial sequence
  Synthetic peptide
MADVFPGNDSTASQDVANRFARKGALRQKN

SEQ ID NO: 76 - protein, 30 aa, Artificial sequence
  Synthetic peptide
MADPAAGPPPSEGEESTVRFARKGALRQKN

SEQ ID NO: 77 - protein, 29 aa, Artificial sequence
  Synthetic peptide
MAGLGPGVGDSEGGPRPLFCRKGALRQKV

SEQ ID NO: 78 - protein, 50 aa, Artificial sequence
  Synthetic peptide
EIWLELKPQGRMLMNARYFLEMSDTKDMNEFETEGFFALHQRRGAIKQAK

SEQ ID NO: 79 - protein, 50 aa, Artificial sequence
  Synthetic peptide
EFWLDLQPQAKVLMSVQYFLEDVDCKQSMRSEDEAKFPTMNRRGAIKQAK

SEQ ID NO: 80 - protein, 50 aa, Artificial sequence
  Synthetic peptide
EDWIDLEPEGRVYVIIDLSGSSGEAPKDNEERVFRERMRPRKRQGAVRRR

SEQ ID NO: 81 - protein, 47 aa, Artificial sequence
  Synthetic peptide
EGWVDLEPEGKVFVVITLTGSFTEATLQRDRIFKHFTRKRQRAMRRR

SEQ ID NO: 82 - protein, 42 aa, Artificial sequence
  Synthetic peptide
PAAGPPPSEGEESTVRFARKGALRQKNVHEVKNHKFTARFFK

SEQ ID NO: 83 - protein, 42 aa, Artificial sequence; identical to SEQ ID NO: 82
  Synthetic peptide

SEQ ID NO: 84 - protein, 42 aa, Artificial sequence
  Synthetic peptide
VFPGNDSTASQDVANRFARKGALRQKNVHEVKDHKFIARFFK

SEQ ID NO: 85 - protein, 41 aa, Artificial sequence
  Synthetic peptide
LGPGVGDSEGGPRPLFCRKGALRQKVVHEVKSHKFTARFFK

SEQ ID NO: 86 - protein, 60 aa, Artificial sequence
  Synthetic peptide
LQPQAKVLMSVQYFLEDVDCKQSMRSEDEAKFPTMNRRGAIKQAKIHYIKNHEFIATFFG

SEQ ID NO: 87 - protein, 60 aa, Artificial sequence
  Synthetic peptide
LKPQGRMLMNARYFLEMSDTKDMNEFETEGFFALHQRRGAIKQAKVHHVKCHEFTATFFP

SEQ ID NO: 88 - protein, 60 aa, Artificial sequence
  Synthetic peptide
LEPEGRVYVIIDLSGSSGEAPKDNEERVFRERMRPRKRQGAVRRRVHQVNGHKFMATYLR

SEQ ID NO: 89 - protein, 57 aa, Artificial sequence
  Synthetic peptide
LEPEGKVFVVITLTGSFTEATLQRDRIFKHFTRKRQRAMRRRVHQINGHKFMATYLR

SEQ ID NO: 90 - protein, 48 aa, Artificial sequence
  Synthetic peptide
LKAHYGGDIFITSVDAATTFEELCEEVRDMCRLHQQHPLTLKWVDSEG

SEQ ID NO: 91 - protein, 43 aa, Homo sapiens
  portion of epsilon PKC
PEGRVYVIIDLSGSSGEAPKDNEERVFRERMRPRKRQGAVRRR

SEQ ID NO: 92 - protein, 43 aa, Oryctolagus cuniculus
  portion of epsilon PKC
PEGKVYVIIDLSGSSGEAPKDNEERVFRERMRPRKRQGAVRRR

SEQ ID NO: 93 - protein, 43 aa, Rattus norvegicus; identical to SEQ ID NO: 92
  portion of epsilon PKC

SEQ ID NO: 94 - protein, 43 aa, Mus musculus; identical to SEQ ID NO: 92
  portion of epsilon PKC

SEQ ID NO: 95 - protein, 24 aa, Artificial sequence
  Synthetic peptide
DLSGSSGEAPKDNEERVFRERMRP